(19) United States
(12) Patent Application Publication
(10) Pub. No.: US 2019/0112580 A1
(43) Pub. Date: Apr. 18, 2019

(54) METHODS FOR TREATING CONTRACTURED MUSCLE AND RELATED CULTURED CELLS

(71) Applicant: Rehabilitation Institute of Chicago d/b/a Shirley Ryan AbilityLab, Chicago, IL (US)

(72) Inventors: Andrea Alberto DOMENIGHETTI, Chicago, IL (US); Richard L. LIEBER, Chicago, IL (US)

(73) Assignee: Rehabilitation Institute of Chicago d/b/a Shirley Ryan AbilityLab, Chicago, IL (US)

(21) Appl. No.: 16/161,468

(22) Filed: Oct. 16, 2018

Related U.S. Application Data
(60) Provisional application No. 62/572,609, filed on Oct. 16, 2017.
(60) Provisional application No. 62/640,761, filed on Mar. 9, 2018.

(51) Int. Cl. C12N 5/071; A61P 25/14; C12N 5/077; C12N 5/00; A61K 38/00; A61K 31/45; A61K 31/7115; A61K 31/52; A61K 31/7068

(52) U.S. Cl. CPC C12N 5/0697; A61P 25/14; C12N 5/0659; C12N 5/0018; A61K 31/7068; A61K 31/45; A61K 31/7115; A61K 31/52; A61K 38/005

(57) ABSTRACT

This disclosure relates to the use of DNA methyltransferase inhibitors, alone or in combination with Aurora kinase inhibitors, to treat muscle cells isolated from contractured muscles or to prepare isolated and treated muscle cell compositions. The analogs or drug combinations or cell compositions can be used to treat or to prevent skeletal muscle contractures, skeletal muscle atrophy, and muscle tissue remodeling in patients such as those with cerebral palsy. A method is provided for the treatment of the conditions listed above using muscle cells treated with DNA methyltransferase inhibitors in vitro, alone or along with other active agents, cells or biocompatible materials, and transplanted into patients with cerebral palsy or other muscle conditions.

### CROSS-REFERENCE TO RELATED APPLICATIONS

This application claims priority to U.S. Provisional Patent Application No. 62/572,609 filed on Oct. 16, 2017 and U.S. Provisional Patent Application No. 62/640,761 filed on Mar. 9, 2018. The entire disclosure of each of the above-referenced disclosures is specifically incorporated herein by reference.

### REFERENCE TO A SEQUENCE LISTING

The instant application contains a Sequence Listing, which has been submitted in ASCII format via EFS-Web and is hereby incorporated by reference in its entirety. Said ASCII copy, created on Oct. 12, 2018, is named P4892US00_582150 ST25.txt and is 1607 bytes in size.

### BACKGROUND

Cerebral Palsy (CP) is a neurologically non-progressive condition caused by a perinatal insult to the brain. While the brain lesion is non-progressive, individuals with CP have dramatic and progressive changes to their skeletal muscles, including changes in connective tissue composition and reduced force-generating capacity secondary to both central and peripheral factors. CP is also associated with development of spasticity (i.e., abnormal increase in muscle tone or stiffness), muscle atrophy (i.e., a decrease in muscle mass), shorter muscles, and progressive development of debilitating muscle contractures (i.e., a tightening or shortening of muscles and tendons), which may manifest as a reduced range of joint excursion. Given the relatively high prevalence of CP (e.g., 2-3 per 1000 births), CP puts a significant and long-lasting economic and emotional burden on patients, families, and society. It is estimated that medical costs for children with CP are 10-26 times higher than for typically developing children. The Centers for Disease Control and Prevention (CDC) also estimated that the lifetime cost to care for an individual with CP was nearly $1 million in 2003 dollars.

Restoring muscle strength and function, while managing spasticity and progression of muscle contractures, are the fundamental goals of rehabilitation and surgery in children with CP. Conservative treatments include physical and occupational therapies, as well as spasticity management using focal or systemic drugs and surgical intervention to release tendons, lengthen muscles, or correct bony deformities. Despite this variety of potential interventions, among children with CP, 1 in 3 cannot walk, and among those who can walk, 1 in 6 regularly require walking aids and may lose the capacity to walk during adolescence. Therefore, there is an urgent need for new innovative therapeutic approaches to improving outcomes and the quality of life in this patient population.

Development of spasticity, muscle atrophy, and contractures also can occur in other chronic conditions, including alternative types of brain injury (e.g., stroke, traumatic brain injury), spinal cord injury, and muscular dystrophy. There is a general need for improved methods and therapies in conditions with contractured muscles.

### BRIEF DESCRIPTION

A method is disclosed for culturing muscle cells isolated from a contractured muscle of a patient comprising culturing isolated patient muscle cells in the presence of a DNA methyltransferase inhibitor in a proliferation medium. The DNA methyltransferase inhibitor may be 5-azacytidine, decitabine, zebularine, caffeic acid, chlorogenic acid, (−)-epigallocatechin gallate, hydralazine, procaine, or N-phthalyl-L-tryptophan (RG108). The isolated patient muscle cells may be cultured in the presence of the DNA methyltransferase inhibitor in the proliferation medium for a period of 1 to 24 hours. The isolated muscle cells may further be cultured in the presence of an Aurora kinase inhibitor.

The method may further comprise removing the isolated patient muscle cells from the DNA methyltransferase inhibitor and culturing the removed isolated patient muscle cells in the presence of an Aurora kinase inhibitor in a proliferation medium.

The isolated muscle cells are cultured in the presence of the Aurora kinase inhibitor after being cultured in the presence of the DNA methyltransferase inhibitor.

The Aurora kinase inhibitor may be 2-(4-morpholinoanilino)-N6-cyclohexyladenine or a 2-(4-morpholinoanilino)-N6-cyclohexyladenine analog.

The DNA methyltransferase inhibitor may be present in the culture for about 6 hours, 24 hours, or 48 hours.

The muscle cells may comprise muscle satellite cells. The muscle cells may comprise myoblasts. The myoblasts may be derived in vitro from muscle satellite cells. The muscle cells may comprise a portion of muscle fiber. The portion of muscle fiber may be dissected. The portion of muscle fiber may be cultured in vitro. The myoblasts may be derived from a muscle fiber. The muscle fiber may be a dissected muscle fiber. The muscle fiber may be cultured in vitro.

The muscle cells may be obtained from the contractured muscle of a patient having cerebral palsy.

The method may comprise culturing the cells in the presence of the DNA methyltransferase inhibitor for about 1 to about 24 hours and further comprising administering an Aurora kinase inhibitor to the isolated muscle cells for up to four days.

The DNA methyltransferase inhibitor used may be 5-azacytidine and the Aurora kinase inhibitor used may be 2-(4-morpholinoanilino)-N6-cyclohexyladenine. The Aurora kinase inhibitor may be applied at about the same time as the DNA methyltransferase inhibitor is applied.

Isolated cells from contractured muscle are further disclosed. The isolated cells from contractured muscle may be obtained from a patient (e.g., a patient having cerebral palsy), wherein the cell has been cultured in a proliferation medium in vitro in the presence of a DNA methyltransferase inhibitor. The cell may also have been cultured in the presence of an Aurora kinase inhibitor. The cell may be differentiated into a myoblast prior to having been cultured in the proliferation medium. The cell may be a satellite cell. The cell may have been cultured concurrently in the presence of the DNA methyltransferase inhibitor and the Aurora kinase inhibitor. The cell may be passaged 1 to 6 times before culturing in the presence of the DNA methyltransferase inhibitor. The cell may be passaged 1 to 6 times before culturing in the presence of the DNA methyltransferase inhibitor and the Aurora kinase inhibitor. The cell may be cryopreserved before culturing in vitro in the presence of a DNA methyltransferase inhibitor. The cell may have been cultured in the presence of the DNA methyltransferase inhibitor for about 1 to about 24 hours and may have been cultured in the presence of an Aurora kinase inhibitor for up to four days.

The isolated cultured muscle cell may be passaged 1 to 6 times. The isolated cultured muscle cell may be cryopreserved.

An isolated cell from cultured muscle is disclosed that may be obtained by the methods disclosed herein. The cell may be obtained by isolating the muscle cell from a contractured muscle of a patient and culturing the isolated patient muscle cells in the presence of a DNA methyltransferase inhibitor in a proliferation medium. The DNA methyltransferase inhibitor may be 5-azacytidine. The isolated muscle cells may be cultured in the presence of the Aurora kinase inhibitor after being cultured in the presence of the DNA methyltransferase inhibitor.

Methods of treating patients using isolated muscle cells are further disclosed. A method for treating a patient having cerebral palsy may comprise the step of administering to the patient the isolated patient muscle cells obtained by of the methods disclosed herein. For example, a method for treating a patient having cerebral palsy may comprise the step of administering to the patient the isolated patient muscle cells obtained from a contractured muscle of a patient, wherein the cells isolated from contractured muscle have been cultured in a proliferation medium in vitro in the presence of a DNA methyltransferase inhibitor.

A method of treating skeletal muscle contractures, skeletal muscle atrophy or remodeling muscle tissue in a patient in need thereof may comprise administering the cells obtained from a contractured muscle of a patient, wherein the isolated cells from contractured muscle have been cultured in a proliferation medium in vitro in the presence of a DNA methyltransferase inhibitor, wherein the cells are administered intramuscularly at least once.

A method of treating skeletal muscle contractures, skeletal muscle atrophy or remodeling muscle tissue in a patient in need thereof may comprise administering the cells obtained from a contractured muscle of a patient, wherein the isolated cells from contractured muscle have been cultured in a proliferation medium in vitro in the presence of a DNA methyltransferase inhibitor, wherein the cells are administered by surgical transplant at least once.

### DESCRIPTION OF THE FIGURES

The patent or application file contains at least one drawing executed in color. Copies of this patent or patent application publication with color drawing(s) will be provided by the Office upon request and payment of the necessary fee.

FIG. 1 is a schematic of in vitro myogenesis and biomarkers that are typically expressed during the different stages of satellite cell proliferation, differentiation, and maturation.

FIG. 2A to 2C depicts that myoblasts derived from satellite cells isolated from contractured muscles in CP patients have decreased capacity to fuse and to produce myotubes in vitro. FIG. 2A depicts immunostaining of myotube cell preparations from a CP patient and from a typical developing subject. The CP and typically developing (TD) cell preparations were differentiated for 42 hours and then stained with MYH7 and DAPI. The CP cell preparations are seen as spindly and thin (upper-right and lower-right images of FIG. 2A) as compared to the TD cell preparations (upper-left and lower-left images of FIG. 2A). The images at the bottom of FIG. 2A are MYH7 stained only.

FIG. 2B displays a graphic quantification of the Fusion Indexes for the CP and TD patient groups.

FIG. 2C displays immunostained myotube cell preparations from one TD patient (two left images) and two CP patients (middle and right images). Cells were differentiated for 42 hours, immunostained for TUBB, and counterstained for membrane marker WGA and nuclear marker with DAPI.

FIG. 3A to 3E shows impaired integrin signaling during myoblast fusion and differentiation in CP. FIG. 3A shows a Western blot depicting differential proteins expression for integrin beta-1D (ITGB1D, bands at ˜116 kDa), focal adhesion kinase (FAK) phosphorylated at Y576-7 residues (FAK Y576-7, ˜125 kDa), FAK phosphorylated at residue Y925 (FAK Y925, 125 kDa), total FAK protein levels (FAK TOT, ˜125 kDa), and the total protein load (bands in the 5-25 kDa range). FIG. 3B displays protein quantification of ITGB1D. FIG. 3C displays protein quantification for FAK Y397, normalized over FAK TOT. FIG. 3D displays protein quantification for FAK Y576-7, normalized over FAK TOT. FIG. 3E displays protein quantification for FAK Y925, normalized over FAK TOT.

FIG. 4A to 4E show the 24-hr treatment of satellite cell-derived myoblast cultures with the DNA methyltransferase inhibitor, 5-azacitidine (5-AZA). FIG. 4 indicates that treatment was sufficient to cause hypomethylation of the CpG island located in the promoter region of the human integrin ITGB1 gene, restoring repressed ITGB1D protein expression levels during myoblast fusion and promoting myotube formation in CP cultures. FIG. 4A is a graphic that displays DNA methylation analysis of the CpG island located in the promoter region of the human ITGB1 gene in differentiating CP and TD myoblasts that were treated with 5-AZA for 24 hours (referred to in FIG. 4A as “CP-AZA”) or not treated (referred to as “CP” and “TD” in FIG. 4A), along with a negative control (referred to as “Neg” in FIG. 4A).

FIG. 4B displays immunofluorescence staining for TD and CP myotube preparations formed from myoblasts that had been treated with 5-AZA (indicated on FIG. 4B as “+AZA”) or not treated with 5-AZA.

FIG. 4C displays the quantification of Fusion Indexes for CP and TD derived myoblasts that were treated with 5-AZA or not treated.

FIG. 4D displays protein quantification for ITGB1D isoform by Western blotting in non-treated (“CP”) and treated (“CP+AZA”) myoblast cultures after 42-hour differentiation.

FIG. 4E reports a representative Western blot depicting differential expression of integrin beta-1D (ITGB1D, bands at ˜116 kDa), in CP preparations either non-treated (referred to in FIG. 4E as “CP”) or treated with 5-AZA (referred to in FIG. 4E as “CP-AZA”).

FIG. 5, from left to right, depicts 5 columns with 5 different assays on myotube preparations differentiated from the myoblast cell preparations separated into five dishes. The first column reflects myotubes differentiated from the myoblast cell preparation from the first dish, which was not exposed to 5-AZA or reversine. The second column reflects myotubes differentiated from the myoblast cell preparation from the second dish, which was exposed to 5 μM of 5-AZA in high serum medium for 6 hours. The third column reflects myotubes differentiated from the myoblast cell preparation from the third dish, which was exposed to 5 μM of 5-AZA in high serum medium for 24 hours. The fourth column reflects myotubes differentiated from the myoblast cell preparation from the fourth dish, which was exposed to 5 μM of reversine in high-serum medium for 3 days. The final, fifth column reflects myotubes differentiated from the myoblast cell preparation from the fifth dish, which was exposed to 5 μM reversine and 5 μM of 5-AZA in high serum medium for 24 hours followed by incubating the cells for 3 days with 5 μM reversine only in high serum medium. The upper row of panels displays myotubes that have been immunostained, with green WGA depicting membranes, nuclei in blue using DAPI, and myotubes in red using ACTA2. The middle row (gray scale) depicts the staining of the nuclei only using DAPI. The bottom row shows the gray scale staining for the muscle fibers only using ACTA2.

FIG. 6, from left to right, depicts 5 columns with 5 different assays on a myotube preparation derived from satellite cells isolated from the muscle of a TD subject. The myotubes were grown from myoblasts that had been treated or not treated with 5-AZA and/or reversine. The first column reflects no treatment of the myoblasts with either 5-AZA or reversine. The second column reflects treatment with 5 μM of 5-AZA in high serum medium for 6 hours. The third column depicts treatment of myoblasts with 5 μM 5-AZA in high serum medium for 24 hours. The fourth column depicts treatment of myoblasts with 5 μM of reversine in high-serum medium for 3 days, and the final or fifth column depicts co-treatment of the cells with 5 μM reversine and 5 μM of 5-AZA in high serum medium for 24 hours followed by incubating the cells for 3 days with 5 μM reversine only in high serum medium. The upper row of panels has been immunostained with green WGA depicting membranes, nuclei in blue using DAPI, and myotubes in red using ACTA2. The middle row (gray scale) depicts the staining of the nuclei only using DAPI. The bottom row shows the gray scale staining for the muscle fibers only using ACTA2.

FIG. 7A depicts a graph which plots an iCELLigence-generated Delta Cell Index for cell cultures from a typically developing child (TD) and two children with cerebral palsy (CP1 and CP2). FIG. 7B depicts a chart which compares the average slopes of the plots in FIG. 7A for TD and CP cells.

FIG. 8A and FIG. 8B depict linear regression plots quantifying the correlation between iCELLigence-generated slopes and fusion indices.

FIG. 9A illustrates global methylation levels of DNA in proliferating CP and TD myoblast cultures that were treated or non-treated with 5-AZA. Methylation levels were measured using a MethylFlash Methylated DNA Quantification Kit (EpiGentek, P-1034-96). Myoblasts were seeded into gelatin-coated 10 cm plates. Myoblast cell preparations from CP and TD study subjects were exposed to 5 μM of 5-AZA in proliferation medium. 5-AZA was introduced while the myoblast cell preparations were being developed in the high-serum medium, once the myoblast cell preparations were at about 60% of cell plate confluence by visual inspection. The myoblast cell preparations each were cultured in a high-serum medium in the presence of 5 μM of 5-AZA for 24 hours. The myoblast cell preparations then had the 5-AZA washed off by switching the myoblast cell preparations each to fresh high-serum medium and further expanded to 90% of confluence. DNA was extracted from these cultures using the “DNA Purification from Cultured Cells” protocol as described in the Gentra Puregene DNA isolation kit (Qiagen, 158388).

FIG. 9B. A cell impedance-measuring xCELLigence RTCA DP instrument was used to quantify rates of “cell doubling time” (a measurement of cell proliferation) in CP and TD myoblast preparations, treated and non-treated with 5-AZA, as illustrated in FIG. 9B. Proliferating myoblasts were treated with 5 μM 5-AZA (Sigma-Aldrich) for 24 hours to induce DNA hypomethylation. Other cell preparations were not treated with 5-AZA to serve as a control. Following treatment, myoblasts were harvested from exponential growth phase cultures by a standardized detachment procedure using 0.05% Trypsin-EDTA and counted. Myoblast suspensions were seeded into wells of gelatin-coated electrode array plates (E-plates) at 400 and 800 cells/well. Two replicates of each cell concentration were used in each test. Plates were locked in the RTCA DP device in the incubator and the impedance value of each well was automatically monitored by the xCELLigence system and expressed as a Cell Index value (CI). CP and TD myoblasts were incubated during 10 days in a high-serum medium containing Ham's FIO media (Corning) with 20% Fetal Bovine Serum (FBS, from Gibco), 10 mg/ml streptomycin, 100 units/ml penicillin (Gibco), and human recombinant bFGF (bovine fibroblast growth factor, 5 ng/ml) (BD). CI was recorded every 15 min for the entirety of the period. Cell doubling time was calculated from the exponential phase of the growth curve generated by the CI values using the supplied RTCA software.

### DETAILED DESCRIPTION

A targeted treatment of skeletal muscle contractures, skeletal muscle atrophy, pathologic remodeling of muscle tissue and cells with a DNA methyltransferase inhibitor, optionally in combination with other active agents for said condition, that treats or ameliorates the progressive muscle maladaptation that occurs in CP patients and in patients with other conditions where muscle contractures, skeletal muscle atrophy or pathologic remodeling of muscle tissue and cells occur. The approach could assist with the development of specific and personalized treatment planning for children and adults with CP and other muscle contracture diseases.

Described here is the use of DNA methyltransferase inhibitors, such as 5-azacytidine (5-AZA), and Aurora kinase inhibitors, such as reversine, alone or in combination to promote or to rescue myogenic potential of satellite cells and their derivatives, such as myoblasts, in CP patients and in patients having other conditions wherein muscle contractures, skeletal muscle atrophy, and pathologic muscle tissue remodeling are diagnosed.

The methods and compositions described herein may be used to treat or to prevent or ameliorate skeletal muscle contractures, skeletal muscle atrophy, and pathologic muscle tissue remodeling in a patient population in need thereof, such as in CP patients.

Methods are provided for treating a patient suffering from muscle contractures, skeletal muscle atrophy, and in need of muscle tissue remodeling using muscle satellite cells and their derivatives, wherein these cells are isolated from the patient, and treated with a DNA methyltransferase inhibitor alone or in combination with an Aurora kinase inhibitor. The isolated and treated cells can be injected intramuscularly (i.m.) or transplanted back into the patient from which the cells originated. The injected or surgically transplanted cells may be cells alone or in combination with some medically approved biocompatible surface, material or pharmaceutical agent. The isolated and treated cells may be expanded in vitro and cryopreserved for future use.

The cells described herein may be satellite cells and their derivatives obtained from the patient generally via biopsy. The satellite cell derivatives can be produced during the biological process called myogenesis, including myoblasts, myotubes, and muscle fibers, isolated from a muscle biopsy or generated in vitro as described herein or known in the art. The cells to be injected back into the patient may be satellite cells, myoblasts, or a combination of satellite cells and myoblasts. The cells to be transplanted back into the patient may be satellite cells, myoblasts, myotubes, or a combination of satellite cells, myoblasts, or myotubes, alone or in combination with some medically approved biocompatible surface.

A pharmaceutical composition of a DNA methyltransferase inhibitor, alone or in combination with Aurora kinase inhibitors, can be administered directly to the patient via a localized injection into a specific skeletal muscle (i.e. intramuscular, i.m.) or transplanted into the patient alone or in combination with a medically approved biocompatible surface. Such a phaiinaceutical composition comprising a DNA methyltransferase inhibitor may also be injected into the patient with cells harvested from the patient and previously treated with said DNA methyltransferase inhibitor, alone or in combination with Aurora kinase inhibitors, or may be transplanted into the patient alone or in combination with a medically approved biocompatible surface.

The described pharmaceutical composition of analogs and inhibitors alone or in combination with the treated cells may be provided to patients with CP or other conditions where muscle contractures or skeletal muscle atrophy are diagnosed, such that the molecular and physiological profiles of muscle satellite cells in these patients is restored towards non-pathological levels.

The one or more agents and/or compositions may be applied to cultured cells in vitro. Then the cells may be treated with DNA methyltransferase inhibitor and/or Aurora kinase inhibitors before transplantation into the individual's muscle in vivo. In one method, the cells are transplanted by localized injection into a specific skeletal muscle.

In another method, satellite cells and their derivatives myoblasts, myotubes, and/or muscle fibers, may be applied to a bio-compatible support, including 3D-printed bio-compatible scaffolds or tissue patches, and then locally transplanted with their bio-compatible support during surgical procedures for muscle contractures, muscle injury, skeletal muscle atrophy, and other pathologic muscle tissue remodeling. The cells may be treated prior to being applied to the bio-compatible support. The bio-compatible support may be provided with agents which treat the cells upon their application to the bio-compatible support. The cells may be treated for an additional length of time upon being applied to the bio-compatible support before being locally transplanted to the patient.

The methods may further comprise treating the cells in vitro for a period of time, such as 24 hours. The concentration of the agents may vary. For example, 5 μM of DNA methyltransferase inhibitor may be used. The DNA methyltransferase inhibitor alone may be used to treat the satellite cells. In other embodiments, another active agent, cell, or biocompatible material may be applied in conjunction or coordination with the DNA methyltransferase inhibitor.

Various concentrations of DNA methyltransferase inhibitor may be used in connection with the systems and methods described herein. Concentrations of 5-AZA at or above 50 μM are lethal to cells. Exemplary concentrations for use herein include 0.5-15 μM of 5-AZA (and any integer or 0.1 μM value in between) and further exemplary concentrations may include 1 μM, 3 μM, 5 μM, 7 μM, 10 μM, or 15 μM. Similar concentrations for reversine may also be utilized.

The cytidine analogs 5-AZA and decitabine have been extensively investigated in vitro and in vivo in both preclinical and clinical studies related to treatment of myelodysplastic syndromes. Their pharmacokinetic-pharmacodynamic (PK-PD) profile, toxicity, and activity, alone and in combination with other drugs have been tested on hyperproliferative cells and tissues. 5-AZA and decitabine are thought to exert their antineoplastic effects by hypomethylating DNA and are directly toxic to abnormal or rapidly-dividing hematopoietic cells in bone marrow when given at higher doses (Karahoca et al., Pharmacokinetic and pharmacodynamic analysis of 5-aza-2′-deoxycytidine (decitabine) in the design of its dose-schedule for cancer therapy, Clin Epigenetics, 2013; 5(1):3; Cany et al., Decitabine enhances targeting of AML cells by CD34(+) progenitor-derived NK cells in NOD/SCID/IL2Rg(null) mice, Blood, 2018; 131(2):202-14). Separate studies showed that treatment with 5-AZA or decitabine promoted expression of muscle-specific genes, enhanced myogenesis and myotube formation in mouse-derived C2C12 muscle cell lines (Hupkes et al. Epigenetics: DNA demethylation promotes skeletal myotube maturation, FASEB J, 2011; 25(11):3861-72; Montesano et al., Modulation of cell cycle progression by 5-azacytidine is associated with early myogenesis induction in murine myoblasts, Int J Biol Sci, 2013; 9(4):391-402; Lee et al., Methylation of the mouse DIx5 and Osx gene promoters regulates cell type-specific gene expression, Mol Cells, 2006; 22(2):182-8), and stem cells isolated from different tissues, including dental pulp (Nakatsuka et al., 5-Aza-2′-deoxycytidine treatment induces skeletal myogenic differentiation of mouse dental pulp stem cells, Arch Oral Biol, 2010; 55(5):350-7; Jung et al., Local myogenic pulp-derived cell injection enhances craniofacial muscle regeneration in vivo, Orthod Craniofac Res, 2017; 20(1):35-43), human amniotic fluid (Ma et al., Clone-derived human AF-amniotic fluid stem cells are capable of skeletal myogenic differentiation in vitro and in vivo, J Tissue Eng Regen Med, 2012; 6(8):598-613; Bossolasco et al., Molecular and phenotypic characterization of human amniotic fluid cells and their differentiation potential, Cell Res, 2006; 16(4):329-36), murine bone marrow (Belema Bedada et al., Activation of myogenic differentiation pathways in adult bone marrow-derived stem cells, Mol Cell Biol, 2005; 25(21):9509-19), and human adipose tissue (Eom et al., Effective myotube formation in human adipose tissue-derived stem cells expressing dystrophin and myosin heavy chain by cellular fusion with mouse C2C12 myoblasts, Biochem Biophys Res Commun, 2011; 408(1):167-73). Concentrations used to stimulate the pro-myogenic program in vitro ranged from 3-20 μM 5-AZA for up to 10 days, and 0.1-100 μM decitabine for up to 6 days. Montesano et al. reported that 5 μM 5-AZA for 3 days was the optimal protocol that induced DNA hypomethylation and improved differentiation of C2C12 myoblasts without overt signs of cytotoxicity. Fan et al. reported a positive myogenic response, with no change in cell viability, after exposing porcine satellite cells to 10 μM decitabine for 48 hours (Fan et al., Sulforaphane causes a major epigenetic repression of myostatin in porcine satellite cells, Epigenetics, 2012; 7(12):1379-90). Thus, a DNA methyltransferase may be used at a concentration in the range of 0.1-100 μM in a proliferation or culturing medium as disclosed herein.

The DNA methyltransferase inhibitor may be applied for different periods. Periods between 6 hours and 24 hours may be utilized (e.g., 8, 10, 12 and other values in between). Periods shorter than six hours may also be appropriate (e.g., 1, 2, 3, 4, and 5 hours).

The cells may be grown in a proliferation medium for the amount of time necessary to have the cell cultures reach 50-80% confluence, wherein the cells can be cultured for one to four days.

Reversine can be applied for a few days, and 5-AZA may be applied for less than 24 hours. Therefore, the combination of both active agents to the cells may be for up to 24 hours together, present in a proliferation medium. The treated cells, which may be myoblasts or a mix of myoblasts and satellite cells, can be administered (injected or surgically transplanted) back into the patient or frozen (cryopreserved) for future use.

### Abbreviations and Definitions

As used in the specification and embodiments, the singular forms “a,” “an” and “the” include plural references unless the context clearly dictates otherwise. For example, the term “a cell” includes a plurality of cells, including mixtures thereof. Similarly, use of “a compound” for treatment or preparation of medicaments as described herein contemplates using one or more compounds of this invention for such treatment or preparation unless the context clearly dictates otherwise.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention.

The term “about” in relation to a reference numerical value, and its grammatical equivalents as used herein, can include the reference numerical value itself and a range of values plus or minus 10% from that reference numerical value. For example, the term “about 10” includes 10 and any amounts from and including 9 to 11. In some cases, the term “about” in relation to a reference numerical value can also include a range of values plus or minus 10%, 9%, 8%, 7%, 6%, 5%, 4%, 3%, 2%, or 1% from that reference numerical value. In some embodiments, “about” in connection with a number or range measured by a particular method indicates that the given numerical value includes values determined by the variability of that method.

“Aurora kinase inhibitor” is meant to include reversine. Reversine is a potent human A3 adenosine receptor antagonist with a Ki of 0.66 μM, and a pan-Aurora A/B/C kinase inhibitor with IC50 of 12 nM/13 nM/20 nM, respectively. Other Aurora kinase inhibitors include reversine, GSK1070916, GSK LIG6, GSK LIG23, PHA-739358, PHA-680632, CYC116, SNS-314, VX-680 (E. Harrington, et al., “VX-680, a potent and selective small molecule inhibitor of the Aurora kinases, suppresses tumor growth in vivo,” Nature Med. 10: 262-7, 2004), AT9283, R763, PF-03814375, AMG-900, MLN8237, ENMD-2076, MK-5108, AZD1152, VE-465, JNJ-770662, CCT129202, AKI-001, hesperadin (S. Hauf, et al., “The small molecule Hesperadin reveals a role for Aurora B in correcting kinetochore-microtubule attachment and in maintaining the spindle assembly checkpoint,” J. Cell Biol. 161: 281-94 (2003)), ZM447439 (B. Bedrick et al., “Aurora Kinase Inhibitor ZM447439 Blocks Chromosome-induced Spindle Assembly, the Completion of Chromosome Condensation, and the Establishment of the Spindle Integrity Checkpoint in Xenopus Egg Extracts,” Mol. Biol. Cell, 16: 1305-18, 2005), BI 811283, CHR-3520, and CTK110 (M. Kollareddy et al. “Aurora kinase inhibitors: progress towards the clinic,” Invest. New Drugs. 30: 2411-32 (2012)).

“Confluence” refers to the percentage of a surface area covered by cells in cell culture.

A “contractured muscle” is a muscle that is tightened or shortened as a result of a medical condition that affects the musculoskeletal system, such as cerebral palsy, stroke, traumatic brain injury, spinal cord injury, or muscular dystrophy.

“Cytosine” is one of the four main bases found in deoxyribonucleic acid (DNA) and ribonucleic acid (RNA), along with adenine, guanine, and thymine (in DNA), and uracil in RNA.

“Cytidine” is a nucleoside molecule that is formed when cytosine is attached to a ribose ring via a β-N1-glycosidic bond.

A “cytidine analog” includes 5-azacytidine (5-AZA) (Vidaza or Azadine) and its analog decytabine. Cytidine analogs also include 5-aza-2′-deoxycytidine (decitabine), 5-aza-2′-deoxy-2′,2′-difluorocytidine, 5-aza-2-40-deoxy-2′-fluorocytidine, 2′-deoxy-2′,2′-difluorocytidine (gemcitabine), cytosine 1-β-D-arabinofuranoside, 2(1H) pyrimidine riboside, 2′-cyclocytidine, arabinofuranosyl-5-azacytidine (fazarabine), dihydro-5-azacytidine, cytosine 1-β-D-arabinofuranoside (ara-C, cytarabine), N4-octadecyl-cytarabine, and elaidic acid cytarabine.

“Differentiation medium” is a cell medium utilized to trigger myoblast fusion and myotube formation.

“DNA methylation” is an epigenetic mechanism that occurs by the addition of a methyl (CH3) group to DNA, thereby often modifying the function of the genes. The most widely characterized DNA methylation process is the covalent addition of the methyl group at the 5-carbon of the cytosine ring resulting in 5-methylcytosine (5-mC). The addition of methyl groups is controlled at several different levels in cells and is carried out by a family of enzymes called DNA methyltransferases (DNMTs).

“DNA methyltransferase inhibitor” means an agent that inhibits the action of any member of the DNA methyltransferase enzyme family. DNA methyltransferase inhibitors include, but are not limited to, cytidine analogs (including 5-azacytidine and decitabine), and non-nucleoside inhibitors such as zebularine, caffeic acid, chlorogenic acid, (−)-epigallocatechin gallate, hydralazine, procaine, or N-phthalyl-L-tryptophan (RG108), including any salts, hydrates, solvates, prodrugs, and any crystal forms in which they may occur.

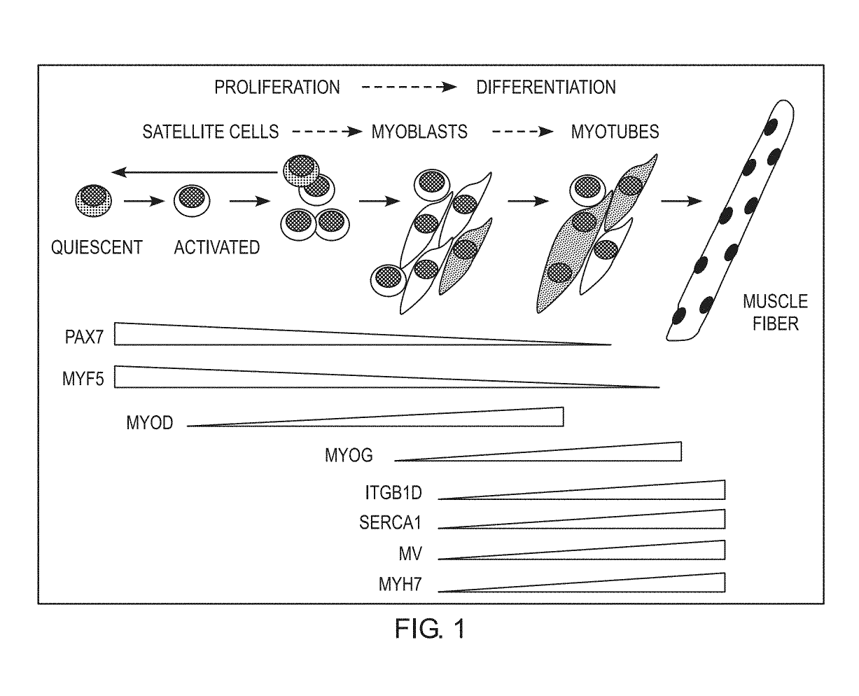

“Myoblast” is a cell, derived from a muscle satellite cell that expresses the biomarker MYOD in a fashion as depicted in FIG. 1.

“Myoblast cell preparation” is a preparation of myoblast cells that may also include, but is not required to also include, muscle satellite cells.

“Myogenesis” is the formation of muscular tissue characterized by satellite cell differentiation into myoblasts, myoblast fusion, and myotube formation.

“Proliferation medium” is a medium conducive to proliferation and expansion of satellite cells and myoblasts.

“Muscle satellite cells” are resident muscle progenitor cells that reside between the basal lamina (a layer of secreted extracellular matrix) and sarcolemma (cell membrane) of muscle fibers. Muscle satellite cells are indispensable for muscle development and repair. Muscle satellite cells are typically defined as mononucleated stem cells normally expressing PAX7 or PAX3 transcription factors, amongst others, which decreases over time as the cells differentiate into myoblasts as depicted in FIG. 1.

A “typically developing” (TD) patient as used in the examples discussed herein is a person eighteen years of age or younger without cerebral palsy or other condition linked to contractured muscles.

### Abbreviations

5-AZA 5-azacitidine

BSA bovine serum albumin

CP cerebral palsy

DMEM Dulbecco's Modified Eagle Medium

DMSO dimethylsulfoxide

DNA deoxyribonucleic acid

DNMT DNA methyltransferase

FACS fluorescence-activated cell sorting

FBS fetal bovine serum

i.m. intramusclularly

ITGB1D Integrin Beta 1D

MV Metavinculin

MYF5 Myogenic Factor 5

MYH7 Slow Myosin Heavy Chain

MYOD Myogenic Differentiation

MYOG Myogenin

MOPS 3-(N-morpholino)propanesulfonic acid)

PAX3 Paired Box 3

PAX7 Paired Box 7

PBS Phosphate buffered saline

PCR Polymerase chain reaction

RIPA Radioimmunoprecipitation assay

RNA ribonucleic acid

SDS sodium dodecyl sulfate

SERCA1 SR Ca (2+)-ATPase 1

TD typically developing

Muscle satellite cells are quiescent in normal post-natal muscle and can be activated by environmental cues, for example, inflammation, damage, disease and growth. Once activated, they divide to produce satellite cell-derived myoblasts that further proliferate and change shape, before fusing to form multinucleated myotubes. The myotubes then mature into contracting muscle fibers. Satellite cells can also divide asymmetrically to produce a self-renewing quiescent satellite cell that will replenish the pool and the niche where it resides.

In muscle tissue, when muscle injury or a pathologic condition occurs, muscle satellite cells are mobilized, proliferate, differentiate into mononucleated myoblasts, and then fuse together to form polynucleated myotubes. Myotubes ultimately become new contracting muscle fibers and replace damaged muscle tissue. In absence of injury or a pathologic condition, muscle satellite cells are important for the physiological growth of muscles during pre- and postnatal development.

Cultured mononucleated satellite cells and their differentiation into fusion-competent myoblasts and then into multinucleated precursors of muscle fibers, called myotubes, provide an excellent in vitro model to study postnatal muscle tissue development or regeneration, and to expand muscle cells for therapeutic purposes.

FIG. 1 displays a schematic of in vitro myogenesis and biomarkers that are typically expressed during the different stages of proliferation, differentiation, and maturation of the muscle satellite cells. The muscle satellite cells are muscle progenitor cells that reside in their niche between the muscle fibers and their basal lamina (connective tissue). Muscle satellite cells play an important role in postnatal muscle growth, repair, and regeneration. Muscle satellite cells can also be identified by expression of, but not limited to PAX7 and PAX3. The bar charts below the images of the cells in FIG. 1 reflect the concentration of various biomarkers through the myogenic differentiation process.

Isolate Satellite Cells from Muscle Biopsies

Satellite cells may be isolated from muscle biopsies obtained during surgical procedures. Different types of surgical procedures may be used to obtain appropriate muscle biopsies. As one example, children with CP can undergo a hamstring lengthening surgery, in which the gracilis and semitendinosus muscles are lengthened to release muscle contractures. TD children without history of neurological disorders may undergo anterior cruciate ligament (ACL) reconstructive surgery using a hamstring autograft.

The cells can then be isolated after biopsy using various techniques. The following description is of enzymatic digestion of the biopsied tissue followed by fluorescent activated cell sorting (FACS). Other methods are known in the art for isolating cells after biopsy, such as enzymatic digestion followed by differential centrifugation, pre-plating on tissue culture plates, or Percoll gradient. Methods may be utilized even in other animal species.

Muscle biopsies can be placed in a container containing a digestion buffer. For example, the muscle biopsies may be placed in a 15 mL conical vial containing 6 mL of digestion buffer (Dulbecco's modified Eagle medium [DMEM; Gibco/Invitrogen, Carlsbad, Calif., USA]; 20 mg/mL collagenase type 1 [Sigma, St. Louis, Mo., USA]; 6 mg/mL dispase II [Roche, Mannheim, Germany]; 6 ku/mL penicillin [Gibco/Invitrogen]; 5 mg/mL streptomycin [Gibco/Invitrogen]). The biopsied tissue can then be transported on ice to a laboratory, and placed in an incubator for a period of time. For example, the biopsied tissue can be placed in an incubator for 30 minutes, at 5% CO₂ and 37° C. Samples can be transferred to tissue culture plates and dissected or disaggregated. For instance, the samples can be dissected using surgical scissors in a sterile tissue culture hood. After the tissue is adequately dissected, it can be returned to the incubator for an additional period of time. For example, it can be returned to the incubator for 50 minutes. The plate and the mixture can then be manually triturated for further mechanical disaggregation of the cells. For instance, the plate and the mixture can be manually triturated through a 5 mL plastic serological pipette. The resulting cell mixture can be incubated for an additional period, such as 5 minutes.

Following incubation, the cell mixture can be filtered through a strainer, such as a 70 μm mesh cell strainer (BD Falcon, San Jose, Calif., USA) into a tube, such as a 50 mL conical-bottom tube. The plate can be subsequently washed with a medium, such as 15 mL of DMEM and 10 mL of fetal bovine serum (FBS; Gibco/Invitrogen) and passed through the filter. The final volume can be brought to an increased volume using DMEM. For example, the final volume may be brought to 40 mL using DMEM. The mixture can then be centrifuged to obtain a pellet containing mononucleated cells. For instance, the mixture may be centrifuged at 600×g for 10 minutes at 4° C. The cell pellet can be resuspended in a buffer, such as 1 mL of fluorescence-activated cell sorting (FACS) buffer (2.5% goat serum; 1 mmol/1 ethylenediaminetetraacetic acid in phosphate-buffered saline [PBS] at pH 7.4).

A sample then can be taken for analysis of total mononuclear cell concentration. For instance, a 15 μL sample can be taken for such analysis. Additional samples can be taken for fluorescence minus one (FMO) controls. For example, 60 μL samples can be taken for such controls. Cell-surface markers (see below) can then be labeled by adding desired conjugated antibodies to the cell suspension. After incubation on ice for a period of time, such as 20 minutes, cells can be pelleted and washed in buffer, such as 3 mL of FACS buffer. Cells can be then washed in FACS buffer, such as 10 mL of FACS buffer and incubated with a secondary antibody on ice for about 20 minutes. Finally, cells can be washed and re-suspended in buffer for analysis. For instance, cells may be washed in 3 mL of FACS buffer and resuspended in 1 mL of FACS buffer for analysis.

Separating Muscle Satellite Cells from Other Cell Populations

Muscle satellite cells can be separated from other cell populations in the muscle tissue using a FACS machine, like the FACSAria II (BD Biosciences, San Jose, Calif., USA). Cells isolated from the muscle biopsy can be stained for cell surface markers, for example by using anti-CD56 antibodies (neural cell adhesion molecule, NCAM, such as clone HCD56) which reliably identifies myogenic PAX7-positive satellite cells, anti-CD31 antibodies (such as clone EP3095), which is a marker of endothelial cells, and anti-CD45 antibodies (such as product Abcam ab10559), which is a marker of leukocytes. Using a FACS machine, CD31 and CD45 marker positive cells can be combined into a dump channel and discarded, while the remaining CD56-positive cells can be collected for cell culture. The resulting satellite cells collected can therefore be defined as CD31-negative/CD45-negative/CD56-positive cells due to the removal of endothelial cells and the leukocytes. This muscle cell sorting procedure can result in the collection of a population of cells committed to undergo myogenesis, rather than neurogenesis, adipogenesis, or other types of cell differentiation. Other cell markers may be used to identify muscle satellite cell populations, including but not limited to the positive selection for markers ITGA7, CD29, and/or CD34, and the negative selection for markers CD11b, ABCG2, and/or SCA1.

The number of satellite cells isolated per mg of tissue biopsy may vary depending on muscle type, muscle biopsy location, and patient age. The number of satellite cells may measure in the thousands to the hundreds of thousands or more depending on the size of the muscle biopsy.

Isolated and sorted satellite cells can be expanded in vitro in a proliferation medium (also referred to herein as a “high serum medium”). One type of proliferation medium is one made of Ham's F10 medium (Corning Life Sciences, Tewksbury, Mass.) combined with 20% Fetal Bovine Serum (“FBS”) (Gibco brand, Thermo Fischer Scientific, Waltham, Mass.) 10 mg/ml streptomycin, and 100 units/ml penicillin (Gibco), human recombinant bFGF (5 ng/ml) (Becton Dickenson, Franklin Lakes, N.J.). The satellite cells may then be frozen in the same proliferation medium, complemented with 10% DMSO (dimethylsulfoxide), at an appropriate concentration, such as 1-2 million cells/mL.

Other proliferation media may be used to expand satellite cells and myoblasts through proliferation in vitro. One example is low-serum (5% or less) or serum-free but chemically defined medium, where the 20% FBS may be replaced with 5% FBS, Fetuin (bovine) 50 μg/ml, Epidermal Growth Factor (EGF, recombinant human) 10 ng/ml, basic Fibroblast Growth Factor (recombinant human bFGF) 1 ng/ml, insulin (recombinant human) 10 μg/ml, dexamethasone 0.4 μg/mL and/or transferrin 30 μg/mL, in combination with a salt-balanced solution, including for example Ham's F12, Ham's F10, or DMEM.

Cells in the proliferation medium may be expanded or sub-cultured (i.e. passaged), for instance in gelatin-, collagen- or laminin-coated tissue culture dishes. In a preferred method, the cells are not passaged more than five or six times under physiological conditions. Adhering cell cultures can be passaged using a 0.4% trypsin solution to detach cells from the culture dish. Cells can be cultured at 37° C. in a 5-10% CO₂ atmosphere using a proliferation medium.

The resulting cell mixture is a myoblast cell preparation, which is a preparation of myoblast cells that may also include some residual muscle satellite cells.

Culturing Isolated Patient Muscle Cells in the Presence of a DNA Methyltransferase Inhibitor

The myoblast cell preparation may be cultured in a proliferation medium in the presence of a DNA methyltransferase inhibitor, such as 5-AZA. A DNA methyltransferase inhibitor may be introduced while the myoblast cell preparation is being developed in the proliferation medium. For instance, a DNA methyltransferase inhibitor may be introduced once the myoblast cell preparation is at 50-80% cell confluence by visual inspection. A higher confluence percentage or density may be appropriate, so long as the confluence is not so high that the cells become overcrowded or grow in multiple layers, resulting in spontaneous cell fusion and/or cell death. The DNA methyltransferase inhibitor may be introduced earlier, for instance, at the same time the isolated satellite cells are added to the proliferation medium. However, the DNA methyltransferase inhibitor should not be introduced in a non-proliferation medium, such as a low-serum differentiation medium, since in such a low-serum differentiation medium it was found that the DNA methyltransferase inhibitor is not conducive to myotube formation by the myoblast cell preparation. Concentrations of DNA methyltransferase inhibitor used may vary. For example, the myoblast cell preparation may be cultured in proliferation medium in the presence of 5 μM of 5-AZA. Other concentrations may be used, although it was determined that 50 μM of 5-AZA is too concentrated and causes the death of the cells in the myoblast cell preparation, and 0.5 μM of 5-AZA is not sufficiently concentrated to cause appropriate myotube growth. The myoblast cell preparation may be cultured in the presence of a DNA methyltransferase inhibitor for a period of time. One appropriate period of time using 5 μM of 5-AZA is 24 hours. As few as 6 hours may be appropriate using 5 μM of 5-AZA. Culturing the myoblast cell preparation in the presence of a DNA methyltransferase inhibitor for more than 24 hours may be acceptable, although there may not be additional beneficial muscle formation by extending the duration of exposure to the DNA methyltransferase inhibitor. Once the myoblast cell preparation has been exposed to the DNA methyltransferase inhibitor for the appropriate duration, the DNA methyltransferase inhibitor may be washed off of the myoblast cell preparation. For example, the myoblast cell preparation may be switched to another medium that does not contain 5-AZA, such as a separate proliferation medium.

Culturing Isolated Patient Muscle Cells in the Presence of an Aurora Kinase Inhibitor

For at least a period of time in which the myoblast cell preparation are cultured in a proliferation medium in the presence of DNA methyltransferase inhibitor, the myoblast cell preparation may also be cultured in the presence of an Aurora kinase inhibitor. One such Aurora kinase inhibitor is reversine.

The DNA methyltransferase inhibitor and the Aurora kinase inhibitor may both be applied to the myoblast cell preparation in the proliferation medium at the same time. After a period of exposure of the DNA methyltransferase inhibitor, the DNA methyltransferase inhibitor may be removed from the myoblast cell preparation while the Aurora kinase inhibitor remains in the presence of the myoblast cell preparation. For example, the DNA methyltransferase inhibitor and the Aurora kinase inhibitor could both be applied to the myoblast cell preparation in the proliferation medium, and then after a period of 6 to 24 hours, the DNA methyltransferase inhibitor could be removed while the Aurora kinase inhibitor remains. Various methods may be used to accomplish the removal. For instance, the myoblast cell preparation may be washed of both the DNA methyltransferase inhibitor and the Aurora kinase inhibitor, and then the Aurora kinase inhibitor may be re-applied to the myoblast cell preparation. Alternately, the myoblast cell preparation may be transferred to a new proliferation medium containing Aurora kinase inhibitor but not DNA methyltransferase inhibitor. For instance, a cell suspension of the myoblast cell preparation that is floating in the proliferation medium may be pelleted in a tube by centrifugation, and the cells may then be cultured in a new proliferation medium.

After removal of the DNA methyltransferase inhibitor, the Aurora kinase inhibitor may remain in the presence of the myoblast cell proliferation in the proliferation medium for a period of time. For example, the Aurora kinase inhibitor may remain for a number of days, such as 2 or 3 days.

As another example, the Aurora kinase inhibitor may be applied sequentially to the DNA methyltransferase inhibitor. For instance, the myoblast cell preparation may be cultured in the presence of the DNA methyltransferase inhibitor for a first period of time, such as between 6 and 25 hours. After the first period of time, the DNA methyltransferase inhibitor would be removed from the myoblast cell preparation. The Aurora kinase inhibitor would then be applied to the myoblast cell preparation for a second period of time, such as for two to three days.

Different concentrations of Aurora kinase inhibitor may be used. For instance, 5 μM of reversine may be used.

Injecting or Transplanting the Isolated Patient Muscle Cells

Myoblast cell preparation that has been exposed to the DNA methyltransferase inhibitor and optionally the Aurora kinase inhibitor the cells may be detached from the culture dish and prepared for injection into muscle tissue or for transplantation onto a bio-compatible surface. The number of cells to be injected or transplanted may range from 10,000 cells per procedure to tens of millions of cells per procedure.

Exposed Myoblast Cell Preparation that has been Exposed to DNA

methyltransferase inhibitor and optionally to Aurora kinase inhibitor may be frozen for later use. For instance, it may be frozen in 2 mL cryotubes (Nalgene), using a controlled rate freezing process. The process may include freezing the cells down, at a rate of 1-2° C./min up to a temperature point of about −80° C. The freezing may occur in a dedicated freezer. Frozen cell preparations may then be moved and stored elsewhere, for instance in liquid nitrogen-filled tanks, until needed for future use. Frozen myoblast cell preparation can be thawed and then injected or transplanted into a patient.

### EXAMPLES

Example 1 and Example 2 compare differences observed in myotube formation using myoblasts derived from muscle satellite cell biopsies obtained from typically developing children (TD) and children with cerebral palsy (CP). In these examples, the cell preparations were not treated with a DNA methyltransferase inhibitor but show differences in cell differentiation between TD and CP patients. Example 3 introduces the effect of treatment of CP and TD myoblasts with the DNA methyltransferase inhibitor 5-AZA before myotube formation. Examples 4 and 5 each support data obtained in Example 3 and introduce the effect of separate or combined treatment of CP and TD myoblasts with 5-AZA and reversine before myotube formation. More specifically, Example 4 and 5 involve a qualitative comparison of (a) myotubes and nuclei density derived from an untreated myoblast cell preparation with (b) myotubes and nuclei density derived from a myoblast cell preparation treated with 5-AZA for six hours; with (c) myotubes and nuclei density derived from a myoblast cell preparation treated with 5-AZA for 24 hours; with (d) myotubes and nuclei density derived from a myoblast cell preparation treated with reversine only; and with (e) myotubes and nuclei density derived from cells treated with a combination of 5-AZA and reversine. Myotube sizes and thickness was analyzed in order to determine the efficacy of having treating isolated satellite cells with various agents, either alone or in combination.

Myotubes were differentiated using the following methods. Once the appropriate level of cell confluence of 50-80% was obtained for the myoblast cell preparation, fusion and differentiation of myoblasts into myotubes was promoted by switching from a high-serum medium to a low-serum differentiation medium. The switch from a high-serum medium to a low-serum differentiation medium is a known potent trigger for myoblasts to fuse and form multinucleated myotubes in vitro. Specifically, the high-serum proliferation medium was made of Ham's F10 medium (Corning Life Sciences, Tewksbury, Mass.) combined with 20% Fetal Bovine Serum (“FBS”) (Gibco brand, Thermo Fischer Scientific, Waltham, Mass.) 10 mg/ml streptomycin, and 100 units/ml penicillin (Gibco), human recombinant bFGF (5 ng/ml) (Becton Dickenson). Specifically, the low-serum differentiation medium was made of DMEM medium (Gibco) containing 2% Horse Serum (HS, Gibco), 4.5 g/L D-Glucose, L-Glutamine, 10 mg/ml streptomycin, 100 units/ml penicillin (Gibco), and 1 μL/mL insulin solution (Sigma-Aldrich). Cell preparations cultured in the low-serum differentiation medium may be analyzed for myotube formation starting from about 24 hours to about 6 weeks after switching from the high-serum to the low-serum medium.

For visual and quantitative analysis by immunostaining, the isolated satellite cell-derived myoblast cell preparations were treated for immunostaining. Cells were rinsed one time (or more) with 1× phosphate buffered saline (“PBS” or “1×PBS”). The cells were then fixed for 10 min with 4% paraformaldehyde, and rinsed three times with 1×PBS. Cells were permeabilized for 10 min. in 1×PBS supplemented with 0.2% Triton X-100 (Sigma-Aldrich, St. Louis, Mo.), washed three times with 1×PBS, and incubated in a blocking solution (150 mM NaCl and 20 mM Tris, pH 7.4) that is supplemented with 1% bovine serum albumin (“BSA”) for 1 hour at room temperature (RT) before incubation with primary antibodies against slow myosin heavy-chain MYH7 (e.g., clone NOQ7.5.4D), α-actinin ACTA2 (e.g., clone EA-53), β-tubulin TUBB (e.g., clone E7). Other desired marker antibodies diluted in blocking solution, at dilutions typically in the range of 1:500 to 1:1000, also could have been used. The fixed cells were incubated with primary antibodies overnight at 4° C. in a 1 ml solution, no shaking required. After incubation, cells were washed three times for 5 min. with 1×PBS and incubated with secondary antibodies (Jackson ImmunoResearch Laboratories, West Grove, Pa.). The secondary antibodies were in a blocking solution for 1 hour at room temperature (“RT”) or overnight at 4° C., at 1:1000 dilution. Secondary antibody mixtures contained 4′,6′-diamidino-2-phenylindol (DAPI), a marker for cell nuclei, and conjugated wheat germ agglutinin (“WGA”) (ThermoFisher Scientific, Waltham, Mass.), and/or a membrane and connective tissue marker. After washing three times with 1×PBS for 5 minutes each to remove the secondary antibody, the cells were mounted using fluorescent mounting medium such as Vectashield® (Vector Laboratories, Burlingame, Calif.). Fluorescence microscopy was performed using an epifluorescence or confocal microscope using the 20× air objective, the 40×, or 63× oil immersion objective and zoom rates between 1 and 3 in sequential scanning mode.

Myoblast fusion and myotube formation can be quantified in vitro using the Fusion Index, which is defined as the total number of nuclei (excluding mono- and binucleated) in MYH7 or ACTA2-positive cells (i.e. myotubes) divided by the total number of nuclei per field. In an embodiment, the Fusion Index of a cell sample may be measured using 5 microscopic fields per 35 mm cell culture dish, acquired on a Leica TCS SP5 confocal microscope using the 20× air objective.

### Example 1

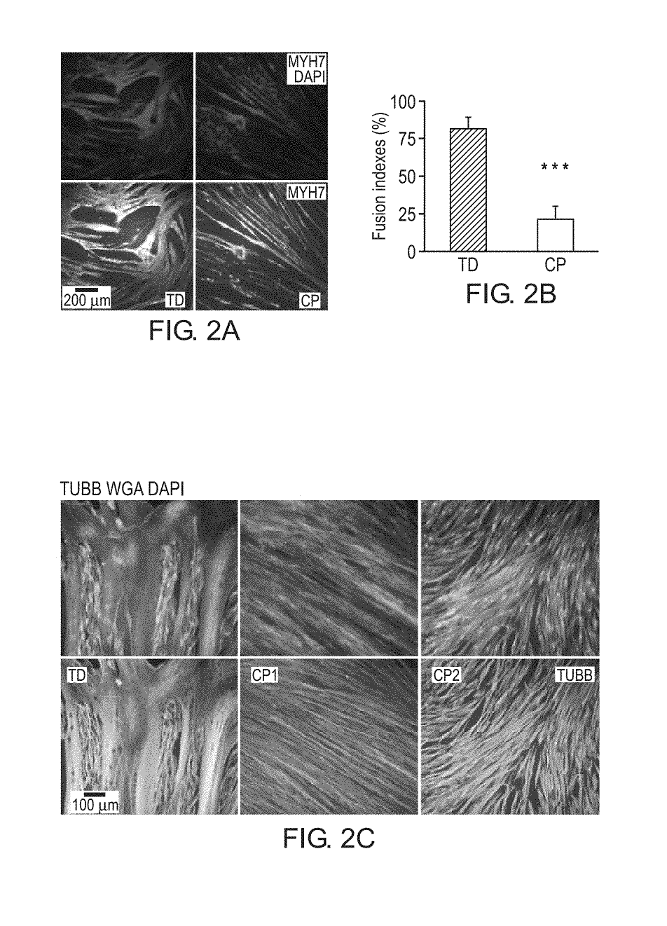

FIG. 2A to 2C show that myoblasts derived from human muscle satellite cells isolated from contractured muscles from a CP patient have a decreased capacity to fuse and to produce myotubes in vitro, which translates into an impaired myogenic potential for these cells. In other words, the cells have a decreased capacity to contribute to the growth and regeneration of muscle tissue in clinical settings.

As used herein, “CP myotubes” means myotubes differentiated from cells taken from the contractured muscle of a patient with CP, and “TD myotubes” means myotubes differentiated from cells taken from the muscle of a TD subject. FIG. 2A displays immunostaining of CP myotube and TD myotubes.

Primary satellite cells were isolated from human muscle, sorted using FACS, plated in gelatin coated dishes, and grown into proliferation medium.

Satellite cells were isolated from muscle biopsies obtained during surgical procedures. Children with CP underwent a hamstring lengthening surgery, in which the gracilis and semitendinosus muscles are lengthened to release muscle contractures. TD children without history of neurological disorders underwent anterior cruciate ligament (ACL) reconstructive surgery using a hamstring autograft.

The cells were isolated after biopsy. The following description is of enzymatic digestion of each biopsied tissue followed by fluorescent activated cell sorting (FACS). Each muscle biopsy was placed in a 15 mL container containing 6 mL of digestion buffer (Dulbecco's modified Eagle medium [DMEM; Gibco/Invitrogen, Carlsbad, Calif., USA]; 20 mg/mL collagenase type 1 [Sigma, St. Louis, Mo., USA]; 6 mg/mL dispase II [Roche, Mannheim, Germany]; 6 ku/mL penicillin [Gibco/Invitrogen]; 5 mg/mL streptomycin [Gibco/Invitrogen]). The biopsied tissue was transported on ice to a laboratory, and placed in an incubator for 30 minutes, at 5% CO₂ and 37° C. Samples were transferred to tissue culture plates and dissected using surgical scissors in a sterile tissue culture hood. After each tissue was adequately dissected, it was returned to the incubator for 50 minutes. The plate and the mixture were manually triturated through a 5 mL plastic serological pipette for further mechanical disaggregation of the cells. Each resulting cell mixture was incubated for an additional 5 minutes.

Following incubation, each cell mixture was filtered through a 70 μm mesh cell strainer (BD Falcon, San Jose, Calif., USA) into a 50 mL conical-bottom tube. Each plate was subsequently washed with 15 mL of DMEM and 10 mL of fetal bovine serum (FBS; Gibco/Invitrogen) and passed through the filter. Each final volume was brought to 40 mL using DMEM. Each mixture was then centrifuged at 600×g for 10 minutes at 4° C. to obtain a pellet containing mononucleated cells. Each cell pellet was re-suspended in a 1 mL fluorescence-activated cell sorting (FACS) buffer (2.5% goat serum; 1 mmol/1 ethylenediaminetetraacetic acid in phosphate-buffered saline [PBS] at pH 7.4).

A 15 μL sample was taken for analysis of total mononuclear cell concentration. Additional 60 μL samples were taken for fluorescence minus one (FMO) controls. Cell-surface markers (see below) were labeled by adding desired conjugated antibodies to the cell suspension. After incubation on ice for 20 minutes, cells were pelleted and washed in 3 mL of FACS buffer. Cells were then washed in 10 mL of FACS buffer and incubated with a secondary antibody on ice for about 20 minutes. Finally, cells were washed in 3 mL of FACS buffer and re-suspended in in 1 mL of FACS buffer for analysis.

Muscle satellite cells were separated from other cell populations in each muscle tissue using the FACSAria II (BD Biosciences, San Jose, Calif., USA). Cells isolated from each of the CP and TD muscle biopsies were stained for cell surface markers using anti-CD56 antibodies (neural cell adhesion molecule, NCAM, clone HCD56) which reliably identifies myogenic PAX7-positive satellite cells, anti-CD31 antibodies (clone EP3095), which is a marker of endothelial cells, and anti-CD45 antibodies (Abcam ab10559), which is a marker of leukocytes. Using the FACS machine, CD31 and CD45 marker positive cells were combined into a dump channel and discarded, while the remaining CD56-positive cells were collected for cell culture. The resulting satellite cells collected were CD31-negative/CD45-negative/CD56-positive cells due to the removal of endothelial cells and the leukocytes. This muscle cell sorting procedure resulted in the collection of a population of cells committed to undergo myogenesis, rather than neurogenesis, adipogenesis, or other types of cell differentiation.

Isolated and sorted satellite cells from each CP and TD study subject were expanded in vitro in a high serum medium made of Ham's F10 medium (Corning Life Sciences, Tewksbury, Mass.) combined with 20% Fetal Bovine Serum (“FBS”) (Gibco brand, Thermo Fischer Scientific, Waltham, Mass.) 10 mg/ml streptomycin, and 100 units/ml penicillin (Gibco), human recombinant bFGF (5 ng/ml) (Becton Dickenson, Franklin Lakes, N.J.). The satellite cells were frozen in the same high serum medium complemented with 10% DMSO (dimethylsulfoxide) at a concentration of 1-2 million cells/mL.

Cells in each proliferation medium were passaged in coated tissue culture dishes. Adhering cell cultures were passaged using a 0.4% trypsin solution to detach cells from the culture dish. Cells were cultured at 37° C. in a 5-10% CO₂ atmosphere using the high-serum medium. Each resulting cell mixture derived from cells from a TD study subject and a CP study subject was a myoblast cell preparation.

The myoblast cell preparations were cultured in the high-serum medium in the presence of the DNA methyltransferase inhibitor 5-AZA. 5-AZA was introduced while the myoblast cell preparations were expanding in the high-serum medium, once the myoblast cell preparations were at about 70% cell confluence by visual inspection. The myoblast cell preparations each were cultured in a high-serum medium in the presence of 5 μM of 5-AZA for 24 hours.

The resulting CP and TD myoblast cell preparations were transferred to a low-serum medium and cultured to differentiate for 42 hours so that myotubes began to form. The resulting cells were immunostained according to the immunostaining methods described above for MYH7 (in red color) and DAPI (blue dots) according to manufacturer's instructions. Compared to the forming TD myotubes, the forming CP myotubes are spindly and thin with both fewer myotubes and nuclei per myotube. The gray scale pictures show MYH7 staining as a single channel, without DAPI staining.

FIG. 2B displays a graphic quantification of Fusion Indexes for CP myoblasts and TD myoblasts. Primary satellite cells from a CP study subject and from a TD study subject were isolated from human muscle, sorted using FACS, plated in gelatin coated dishes, and grown into proliferation medium using the methods described above until a visual confluence of about 70% was reached. The resulting CP and TD myoblast cell preparations were each transferred from the high-serum medium to the low-serum differentiation medium, where the cells were then cultured for an additional 42 hours. The Fusion Index was calculated after the 42-hour period of exposure to the differentiation medium. The Fusion Index reflects how well the myoblast cell preparation fused into myotubes. The data in FIG. 2B demonstrate that there was a statistically significant, approximately 74% decrease of Fusion Index in the CP myoblast cell preparation as compared to the TD myoblast cell preparation. Quantification of the cells was performed after 42 hours of exposure to the differentiation medium and was statistically significant, P<0.001 using a One-Way ANOVA analysis (N=8 biological replicates per group).

FIG. 2C displays immunostaining for the myotube cell preparations differentiated from satellite cells obtained from one TD and two CP patients. Myoblast cell preparations were obtained and then differentiated for 42 hours in the low-serum differentiation medium as discussed above. After 42 hours, the cell population was immunostained as described above for TUBB (red staining), and counterstained for membrane marker WGA (green staining) and nuclear marker DAPI (blue staining). The staining of the differentiated cell preparations shown in FIG. 2C visually confirms impaired cell fusion and impaired myotube formation in CP when compared to TD. The gray scale panels show TUBB immunostainings in a separated single channel without DAPI or WGA. All together, these data suggest that there is a decreased myogenic potential for the satellite cell pool obtained from CP muscles. The decreased myogenic potential could promote conditions harmful to a patient, such as skeletal muscle atrophy, muscle tissue fibrosis, or development of skeletal muscle contractures.

### Example 2

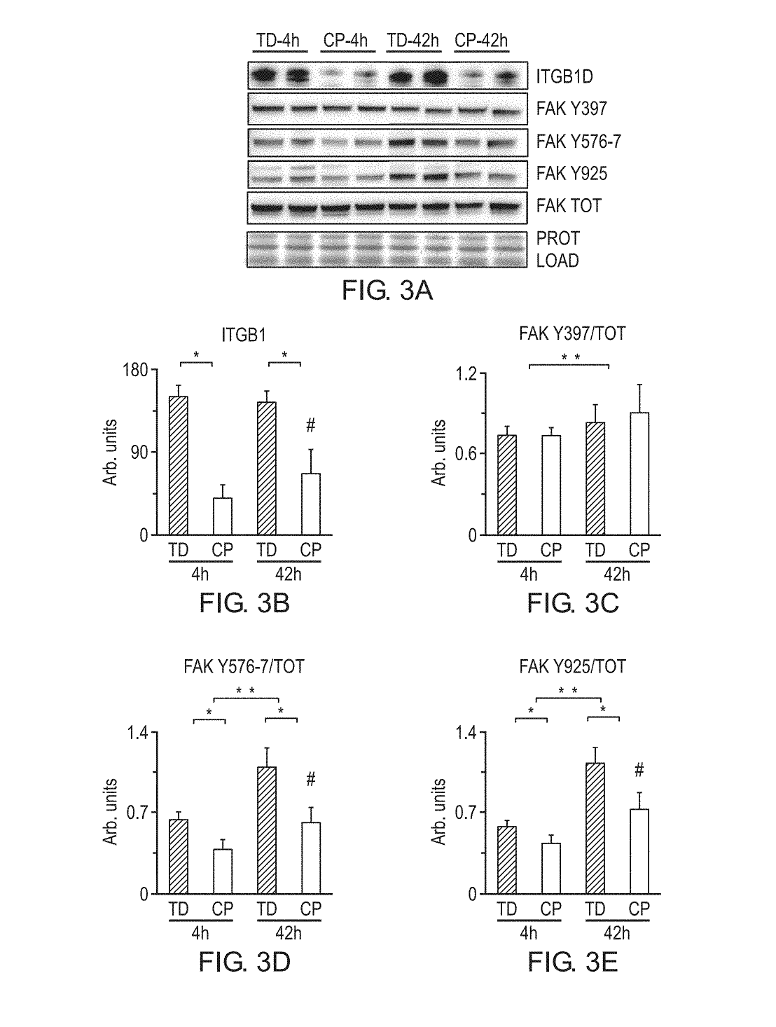

FIG. 3A to 3E demonstrate that decreased ability of myoblasts to fuse and to produce myotubes in vitro is associated in part with downregulation in CP patient-derived cell cultures of the muscle-specific integrin signaling pathway that regulates myoblast fusion during myotube formation. Integrins are heterodimeric transmembrane receptors that mediate cell-extracellular matrix and cell-cell adhesion. They play a significant role in multiple aspects of skeletal muscle homeostasis, including maintenance of the muscle satellite cell niche, myoblast fusion, assembly of costameres and sarcomeres in muscle fibers, development of the myotendinous and neuromuscular junctions, transduction of mechanical signals that lead to cytoskeletal rearrangements and changes in gene expression. In particular, the expression of the ITGB1D protein isoform of the ITGB1 gene is necessary for myoblast fusion and myotube formation during differentiation. One intracellular molecule that mediates integrin receptor signaling is the protein tyrosine kinase, FAK (focal adhesion kinase also known as protein tyrosine kinase 2, PTK2). Phosphorylation of FAK at different tyrosine (Y) amino acid residues is transiently increased during myoblast fusion, and inhibition of this process blocks myotube formation.

Satellite cells were isolated from muscle biopsies obtained during surgical procedures. Children with CP underwent a hamstring lengthening surgery, in which the gracilis and semitendinosus muscles are lengthened to release muscle contractures. TD children without history of neurological disorders underwent anterior cruciate ligament (ACL) reconstructive surgery using a hamstring autograft.

The cells were isolated after biopsy. The following description is of enzymatic digestion of each biopsied tissue followed by fluorescent activated cell sorting (FACS). Each muscle biopsy was placed in a 15 mL container containing 6 mL of digestion buffer (Dulbecco's modified Eagle medium [DMEM; Gibco/Invitrogen, Carlsbad, Calif., USA]; 20 mg/mL collagenase type 1 [Sigma, St. Louis, Mo., USA]; 6 mg/mL dispase II [Roche, Mannheim, Germany]; 6 ku/mL penicillin [Gibco/Invitrogen]; 5 mg/mL streptomycin [Gibco/Invitrogen]). The biopsied tissue was transported on ice to a laboratory, and placed in an incubator for 30 minutes, at 5% CO₂ and 37° C. Samples were transferred to tissue culture plates and dissected using surgical scissors in a sterile tissue culture hood. After each tissue was adequately dissected, it was returned to the incubator for 50 minutes. The plate and the mixture were manually triturated through a 5 mL plastic serological pipette for further mechanical disaggregation of the cells. Each resulting cell mixture was incubated for an additional 5 minutes.

Following incubation, each cell mixture was filtered through a 70 μm mesh cell strainer (BD Falcon, San Jose, Calif., USA) into a 50 mL conical-bottom tube. Each plate was subsequently washed with 15 mL of DMEM and 10 mL of fetal bovine serum (FBS; Gibco/Invitrogen) and passed through the filter. Each final volume was brought to 40 mL using DMEM. Each mixture was then centrifuged at 600×g for 10 minutes at 4° C. to obtain a pellet containing mononucleated cells. Each cell pellet was re-suspended in a 1 mL fluorescence-activated cell sorting (FACS) buffer (2.5% goat serum; 1 mmol/1 ethylenediaminetetraacetic acid in phosphate-buffered saline [PBS] at pH 7.4).

A 15 μL sample was taken for analysis of total mononuclear cell concentration. Additional 60 μL samples were taken for fluorescence minus one (FMO) controls. Cell-surface markers (see below) were labeled by adding desired conjugated antibodies to the cell suspension. After incubation on ice for 20 minutes, cells were pelleted and washed in 3 mL of FACS buffer. Cells were then washed in 10 mL of FACS buffer and incubated with a secondary antibody on ice for about 20 minutes. Finally, cells were washed in 3 mL of FACS buffer and re-suspended in in 1 mL of FACS buffer for analysis.

Muscle satellite cells were separated from other cell populations in each muscle tissue using the FACSAria II (BD Biosciences, San Jose, Calif., USA). Cells isolated from each of the CP and TD muscle biopsies were stained for cell surface markers using anti-CD56 antibodies (neural cell adhesion molecule, NCAM, clone HCD56) which reliably identifies myogenic PAX7-positive satellite cells, anti-CD31 antibodies (clone EP3095), which is a marker of endothelial cells, and anti-CD45 antibodies (Abcam ab10559), which is a marker of leukocytes. Using the FACS machine, CD31 and CD45 marker positive cells were combined into a dump channel and discarded, while the remaining CD56-positive cells were collected for cell culture. The resulting satellite cells collected were CD31-negative/CD45-negative/CD56-positive cells due to the removal of endothelial cells and the leukocytes. This muscle cell sorting procedure resulted in the collection of a population of cells committed to undergo myogenesis, rather than neurogenesis, adipogenesis, or other types of cell differentiation.

Isolated and sorted satellite cells from each CP and TD study subject were expanded in vitro in a high serum medium made of Ham's F10 medium (Corning Life Sciences, Tewksbury, Mass.) combined with 20% Fetal Bovine Serum (“FBS”) (Gibco brand, Thermo Fischer Scientific, Waltham, Mass.) 10 mg/ml streptomycin, and 100 units/ml penicillin (Gibco), human recombinant bFGF (5 ng/ml) (Becton Dickenson, Franklin Lakes, N.J.). The satellite cells were frozen in the same high serum medium complemented with 10% DMSO (dimethylsulfoxide) at a concentration of 1-2 million cells/mL.

Cells in each proliferation medium were passaged in coated tissue culture dishes. Adhering cell cultures were passaged using a 0.4% trypsin solution to detach cells from the culture dish. Cells were cultured at 37° C. in a 5-10% CO₂ atmosphere using the high-serum medium. Each resulting cell mixture derived from cells from a TD study subject and a CP study subject was a myoblast cell preparation.

Proteins were extracted from cells by briefly sonicating samples suspended in a radioimmunoprecipitation assay (“RIPA”) buffer (1.752 g NaCl, 2 mL NP-40, 1 g deoxycholic acid, 1 ml 20% sodium dodecyl sulfate (“SDS”), 6.67 mL 1.5 M Tris at pH 8, to 200 ml with purified water (“ddH₂O”)), followed by 4° C. centrifugation (e.g., using a 10,000×g, table-top centrifuge with Eppendorf FA-45-24-11 rotor (Eppendorf North America, Hauppauge, N.Y.) to remove membrane components. Protein samples were stored in RIPA buffer at −80° C. 15 μg of total proteins were separated using NuPAGE 4-12% Bis-Tris gels (ThermoFisher, Waltham, Mass.) with 20× NuPAGE MES (2-morpholinoethanesulfonic acid) or MOPS (3-morpholinopropane-1-sulfonic acid) SDS running buffers and antioxidants (Novex-Life Technologies). After separation, proteins were transferred to 0.45 μm nitrocellulose membrane in a Tris-glycine buffer complemented with 20% methanol. To visualize bands and detect transfer efficiency, membranes were stained with Ponceau S. Membranes were then blocked in a buffer containing 10 mM Tris, 0.15 M NaCl, 0.1% Tween-20 and 1% BSA and incubated with a desired primary antibody (typically 1:500 to 1:1000 dilution) overnight at 4° C. After washing, membranes were incubated with peroxidase-conjugated secondary antibodies (typically at a 1:1000 dilution) for 1 hour at room temperature (Jackson ImmunoResearch Laboratories). Bandscan was identified using chemiluminescence (ECL). Bands also were analyzed by densitometry on a PXi imager (Syngene) and values expressed as arbitrary values of protein levels, normalized either by total proteins transferred on nitrocellulose membrane (as visualized with Ponceau S) or by another reference protein detected by Western blotting.

Protein analysis by Western blotting showed a significant suppression of ITGB1D levels and FAK phosphorylation at residues Y576-7 and Y925 in CP derived cell cultures after 4 hours and 42 hours of cell cultures being exposed to the low-serum differentiation medium. Time-dependent upregulation of FAK phosphorylation during the cell differentiation culturing was also suppressed in CP derived myoblast cultures as compared to TD derived myoblast cultures.

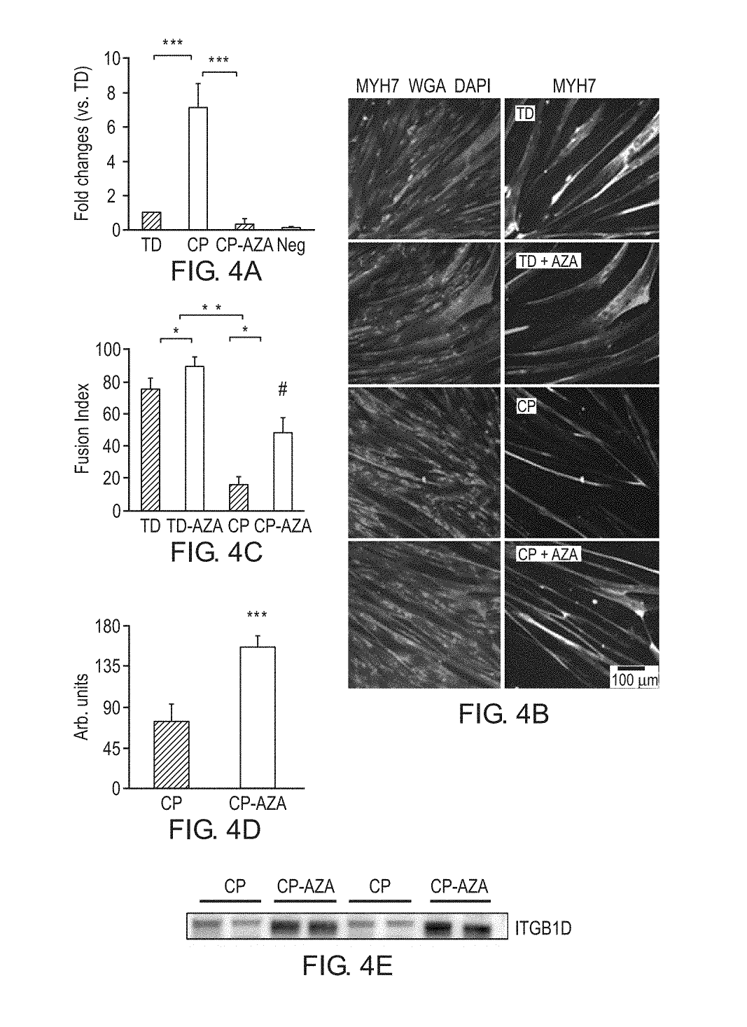

To test for protein expression levels as shown in FIG. 3A to 3E and FIGS. 4D and 4E, Western blotting was used.

FIG. 3A displays representative Western blots showing differential expression for integrin beta-1D (ITGB1D, bands at ˜116 kDa), focal adhesion kinase (FAK) phosphorylated at residue Y397 (FAK Y397, ˜125 kDa), FAK phosphorylated at residues Y576/7 (FAK Y576/7, ˜125 kDa), FAK phosphorylated at residue Y925 (FAK Y925, ˜125 kDa), total FAK protein levels (FAK TOT, ˜125 kDa), and total protein load (bands in the 5-25 kDa range). Protein analysis was performed on the CP and TD cell derived preparations, at 4 hours (4 h) and 42 hours (42 h) after switching the cell cultures from the proliferation medium to the low-serum medium (as described for Example 1).

Primary antibodies used include anti-ITGB1D (obtained from Dr. Robert S. Ross, M.D., University of California San Diego, San Diego, Calif. and as described in C. G. Pham, “Striated muscle-specific beta(1D)-integrin and FAK are involved in cardiac myocyte hypertrophic response pathway,” Am. J. Physiol. Heart. Circ. Physiol. 279(6): H2916-26 (2000)), FAK total and phosphorylated FAK antibodies (Sampler Kit #9330 by Cell Signaling Technology, Inc., Danvers, Mass.).

FIG. 3B displays protein quantification of ITGB1D in CP vs. TD patient derived cells which was statistically significant at P<0.0001 as calculated by Two-Way ANOVA with N=8 per group. Values are obtained for cells tested 4 hours and 42 hours after switching from proliferation medium to low serum medium as described in Example 1.

FIG. 3C displays protein quantification for FAK phosphorylated at residue Y397 and normalized over FAK TOT. Testing was performed at 4 hours and 42 hours after switching from proliferation medium to low serum medium. The comparison was significant at P=0.025 as analyzed using a Two-Way ANOVA (N=6/group).

FIG. 3D displays protein quantification for FAK phosphorylated at residues Y576-7 and normalized over FAK TOT. Testing was performed at 4 hours and 42 hours after switching from high serum to low serum on CP- and TP-patient derived cells. The comparison was significant at P<0.0001 as analyzed using the Two-Way ANOVA (N=6 per group). In CP patient derived cells (#) tested after 42 hours, the significance was P=0.034 as analyzed using the Two-Way ANOVA (N=6/group).

FIG. 4D displays protein quantification for FAK phosphorylated at residue Y925 and as normalized over FAK TOT. CP vs. TD, P<0.0001 for the cells obtain 4 and 42 hours after the high to low serum medium switch. In CP patient derived cells tested after 42 hours, P=0.009, analysis by Two-Way ANOVA (N=6/group).

### Example 3

Changes in DNA methylation on muscle-specific genes, as well as in gene promoters and enhancers regions enriched for CpG dinucleotides (“CpG islands”) play a critical role in regulating activation or inhibition of myogenesis in satellite cells. Integrin beta-1 (ITGB1) gene regulation during the myogenic process is necessary for myoblast fusion and myotube formation. The promoter region of the ITGB1 gene in humans contains a CpG island at position 32957344-32958779 on chromosome 10 (Band: 10p11.22; Genomic Size: 1436; UCSC Genome Browser on Human December 2013 GRCh38/hg38 Assembly).

Satellite cells were isolated from muscle biopsies obtained during surgical procedures. Children with CP underwent a hamstring lengthening surgery, in which the gracilis and semitendinosus muscles are lengthened to release muscle contractures. TD children without history of neurological disorders underwent anterior cruciate ligament (ACL) reconstructive surgery using a hamstring autograft.

The cells were isolated after biopsy. The following description is of enzymatic digestion of each biopsied tissue followed by fluorescent activated cell sorting (FACS). Each muscle biopsy was placed in a 15 mL container containing 6 mL of digestion buffer (Dulbecco's modified Eagle medium [DMEM; Gibco/Invitrogen, Carlsbad, Calif., USA]; 20 mg/mL collagenase type 1 [Sigma, St. Louis, Mo., USA]; 6 mg/mL dispase II [Roche, Mannheim, Germany]; 6 ku/mL penicillin [Gibco/Invitrogen]; 5 mg/mL streptomycin [Gibco/Invitrogen]). The biopsied tissue was transported on ice to a laboratory, and placed in an incubator for 30 minutes, at 5% CO₂ and 37° C. Samples were transferred to tissue culture plates and dissected using surgical scissors in a sterile tissue culture hood. After each tissue was adequately dissected, it was returned to the incubator for 50 minutes. The plate and the mixture were manually triturated through a 5 mL plastic serological pipette for further mechanical disaggregation of the cells. Each resulting cell mixture was incubated for an additional 5 minutes.

Following incubation, each cell mixture was filtered through a 70 μm mesh cell strainer (BD Falcon, San Jose, Calif., USA) into a 50 mL conical-bottom tube. Each plate was subsequently washed with 15 mL of DMEM and 10 mL of fetal bovine serum (FBS; Gibco/Invitrogen) and passed through the filter. Each final volume was brought to 40 mL using DMEM. Each mixture was then centrifuged at 600×g for 10 minutes at 4° C. to obtain a pellet containing mononucleated cells. Each cell pellet was re-suspended in a 1 mL fluorescence-activated cell sorting (FACS) buffer (2.5% goat serum; 1 mmol/1 ethylenediaminetetraacetic acid in phosphate-buffered saline [PBS] at pH 7.4).

A 15 μL sample was taken for analysis of total mononuclear cell concentration. Additional 60 μL samples were taken for fluorescence minus one (FMO) controls. Cell-surface markers (see below) were labeled by adding desired conjugated antibodies to the cell suspension. After incubation on ice for 20 minutes, cells were pelleted and washed in 3 mL of FACS buffer. Cells were then washed in 10 mL of FACS buffer and incubated with a secondary antibody on ice for about 20 minutes. Finally, cells were washed in 3 mL of FACS buffer and re-suspended in in 1 mL of FACS buffer for analysis.

Muscle satellite cells were separated from other cell populations in each muscle tissue using the FACSAria II (BD Biosciences, San Jose, Calif., USA). Cells isolated from each of the CP and TD muscle biopsies were stained for cell surface markers using anti-CD56 antibodies (neural cell adhesion molecule, NCAM, clone HCD56) which reliably identifies myogenic PAX7-positive satellite cells, anti-CD31 antibodies (clone EP3095), which is a marker of endothelial cells, and anti-CD45 antibodies (Abeam ab10559), which is a marker of leukocytes. Using the FACS machine, CD31 and CD45 marker positive cells were combined into a dump channel and discarded, while the remaining CD56-positive cells were collected for cell culture. The resulting satellite cells collected were CD31-negative/CD45-negative/CD56-positive cells due to the removal of endothelial cells and the leukocytes. This muscle cell sorting procedure resulted in the collection of a population of cells committed to undergo myogenesis, rather than neurogenesis, adipogenesis, or other types of cell differentiation.

Isolated and sorted satellite cells from each CP and TD study subject were expanded in vitro in a high serum medium made of Ham's F10 medium (Corning Life Sciences, Tewksbury, Mass.) combined with 20% Fetal Bovine Serum (“FBS”) (Gibco brand, Thermo Fischer Scientific, Waltham, Mass.) 10 mg/ml streptomycin, and 100 units/ml penicillin (Gibco), human recombinant bFGF (5 ng/ml) (Becton Dickenson, Franklin Lakes, N.J.). The satellite cells were frozen in the same high serum medium complemented with 10% DMSO (dimethylsulfoxide) at a concentration of 1-2 million cells/mL.

Cells in each proliferation medium were passaged in coated tissue culture dishes. Adhering cell cultures were passaged using a 0.4% trypsin solution to detach cells from the culture dish. Cells were cultured at 37° C. in a 5-10% CO₂ atmosphere using the high-serum medium. Each resulting cell mixture derived from cells from a TD study subject and a CP study subject was a myoblast cell preparation.

Myoblast cell preparations from CP and TD study subjects were exposed to 5 μM of the DNA methyltransferase inhibitor 5-AZA in proliferation medium. 5-AZA was introduced while the myoblast cell preparations were being developed in the high-serum medium, once the myoblast cell preparations were at about 70% cell confluence by visual inspection. The myoblast cell preparations each were cultured in a high-serum medium in the presence of 5 μM of 5-AZA for 24 hours. The myoblast cell preparations then had the 5-AZA washed off by switching the myoblast cell preparations each to a low-serum medium.

The switch to the low serum containing medium stimulates myoblasts fusion and myotube formation.

Myoblasts were seeded at 50,000 cells/well, at approximately 13,000 cells/cm², in a 12-well plate and maintained in the same proliferation medium as described in Example 1. The cells were allowed to proliferate at 37° C. and 5% CO₂ until they reach up to 50-80% cell confluence by visual inspection. The proliferating myoblasts are then treated with 5 μM 5-AZA (Sigma-Aldrich) for 24 hours to induce DNA hypomethylation before differentiation. 5-AZA was obtained from a 10 uM stock solution diluted 1:2000. In a 2000 mL well, 1 mL of 5 μM 5-AZA was used. Other cells were not treated with 5-AZA to serve as a control. Cell differentiation may be promoted by switching the cells from the proliferation medium to the low-serum medium and culturing the cells in low-serum medium for at least 24 hours at 37° C. and 5% CO₂.

To test for gene promoter methylation levels, a Promoter Methylation PCR Kit (Affymetrix) was be used which includes a DNA methylation binding protein MeCP2/MBP. In this experiment, isolated genomic DNA is digested with MseI restriction enzyme, and the resulting DNA fragments are incubated with the DNA methylation binding protein MeCP2/MBP according to manufacturer's protocol. Methylated DNA fragments were isolated with a spin column and then amplified with promoter specific primers 1FW, 1RV, 2FW, and 2RV encompassing CpG sites. Real-time PCR was used to amplify PCR products. Semi-quantitative PCR could have been used instead of real-time PCR. Likewise, bisulfite bond analysis followed by sequencing or PCR could have been used instead of real-time PCR.

The presence of an amplified product indicates that a specific promoter region is methylated in the isolated genomic DNA sample. The PCR amplification curves or the intensity of the amplified product in the agarose gel following electrophoresis may be used to quantify the levels of DNA methylation for a specific promoter region. Amplification was performed using the materials and according to the methods described below.

FIG. 4A to 4E show that 24-hour treatment of satellite cells-derived myoblast cultures with 5-AZA was sufficient to cause hypomethylation of the CpG island located in the promoter region of the human ITGB1 gene, restoring repressed ITGB1D protein expression levels during myoblast fusion and promoting myotube formation in CP patient derived cell cultures.

FIG. 4A is a graphic that displays DNA methylation analysis of the CpG island located in the promoter region of the human ITGB1 gene in differentiating CP and TD patient derived myoblasts. These cells were either CP myoblasts treated with 5-AZA (referred to in FIG. 4A as “CP-AZA” myoblasts), or non-treated cells from either CP patients (referred to in FIG. 4A as “CP” myoblasts) or TD subjects (referred to in FIG. 4A as “TD” myoblasts. The treated cells were treated with 5 μM 5-AZA for 24 hours.

Two primer pairs targeting different regions of the CpG island in the ITGB1 promoter were designed (as depicted below in at 5′ to 3′ orientation):

(SEQ ID NO: 1)
Primer 1FW: CCTTCGCAGAGGAGGAAACT
(SEQ ID NO: 2)
Primer 1RV: CTCCGGAAACGCATTCCTCT
(SEQ ID NO: 3)
Primer 2FW: GGTAGAAGTTGGCTTAGTGG
(SEQ ID NO: 4)
Primer 2RV: ACGCGGTAAAATGATACTAGAC

These primers encompass DNA regions containing 19 and 15 CpG dinucleotides respectively. An additional primer pair encompassing a region of the promoter without CpG sites was used as a negative control (shown in FIG. 4A as “NEG”) (i.e., Primer 3FW: CAGTTTTCTGTGCTGAGACTGG (SEQ ID NO: 5); Primer 3RV: CCCACTTAATGGATGTTCAAGC (SEQ ID NO: 6)) to quantify DNA methylation level in TD myoblasts. As shown in FIG. 4A, DNA methylation analysis of the CpG island located in the promoter region of the ITGB1 gene showed a ˜7-fold increase in promoter DNA methylation levels in CP patient derived cell cultures after 24 hours of differentiation. A 24-hour treatment of myoblast cultures with 5 μM of 5-AZA before cell differentiation in proliferation medium was sufficient to cause hypomethylation of the CpG island in the ITGB1 promoter region. Analysis was performed semi-quantitatively using “fold-changes” estimates, where the non-treated TD subject cell group (TD) was set to the value of 1. ***TD vs. CP, and ***CP vs. CP-AZA, P<0.001; Analysis performed using One-Way ANOVA (N=4/group).

FIG. 4B displays immunofluorescence staining for TD patient and CP patient derived myotubes cell preparations, of non-treated or treated with 5-AZA (+AZA) cells. Myoblasts were differentiated for 42 hours and then stained for MYH7 (in red color), and counterstained for membrane marker WGA (in green color) and nuclear marker DAPI (in blue color). Gray scale panels (right column of panels in FIG. 4B) show MYH7 staining exported as a single channel. Morphologically, 24 hours of 5 μM 5-azacytidine pretreatment of myoblast cell cultures was sufficient to partially restore myoblast fusion and myotube formation in CP patient derived cells.

FIG. 4C displays the quantification of Fusion Indexes for the CP patient and TD subject derived myoblast cells that are non-treated or treated with 5-AZA (“+AZA” in FIG. 4C). Quantitatively, 24 hours of 5 μM 5-AZA pretreatment of myoblast cell cultures was sufficient to partially restore myoblast fusion and myotube formation in CP patient derived cells, as noted morphologically in FIG. 4B. Quantification for the results depicted in FIG. 4C is performed after 42 hours of cell differentiation as discussed in the examples above. *non-treated vs. 5-AZA treated (+AZA) cells, P<0.0001; **CP vs TD, P<0.0001. In CP patient derived cells treated with 5-AZA, P=0.02, analysis performed by Two-Way ANOVA (N=4/group).

FIG. 4D displays protein quantification for the ITGB1D isoform as analyzed by Western Blotting using the protein quantification methods described above, in non-treated (“CP” in FIG. 4D) and treated (“CP+AZA” in FIG. 4D) CP patient derived myoblast cell cultures after 42-hour differentiation under culturing conditions described above in NuPAGE 4-12% Bis-Tris gels (a gradient gel that goes from 4% to 12%); ***P<0.001, analysis by One-Way ANOVA (N=4).

FIG. 4E reports a representative Western Blots showing differential expression of integrin beta-1D (ITGB1D, bands at ˜116 kDa), in CP preparations non-treated (“CP” in FIG. 4E) or treated with 5-AZA (“CP+AZA” in FIG. 4E). These data show that 5 μM of 5-AZA, applied before differentiation, was sufficient to restore ITGB1D protein expression levels in CP patient derived cell cultures after 48 hours of differentiation in the low serum medium as discussed above.

### Example 4

Reversine is the common name for the 2,6-disubstituted purine derivative 2-(4-morpholinoanilino)-6-cyclohexylaminopurine, also known as 2-(4-morpholinoanilino)-N(6)-cyclohexyladenine 2 or 656820-32-5. Reversine and its 2-substituted adenine derivatives are potent and selective A3 adenosine receptor antagonists. Reversine is also a potent inhibitor of Aurora kinases, which are required for mitotic chromosome segregation, spindle checkpoint function, cytokinesis, histone H3 phosphorylation, chromatin remodeling, and maintenance of the multipotent stage of cells. Reversine may also trigger myoblasts to dedifferentiate into multipotent progenitor-type cells. For these properties, reversine is also labeled as a dedifferentiating agent or molecule. However, it was unclear what reversine alone or in combination with a cytidine analog could do when applied to satellite cell-derived myoblasts from a contractured muscle from a patient such as a CP patient.

This experiment tests treatment of muscle satellite cells and myoblasts with a DNA methyltransferase inhibitor, like 5-azacytidine, in combination with an Aurora kinase inhibitor, such as reversine.

Satellite cells were isolated from muscle biopsies obtained during surgical procedures. Children with CP underwent a hamstring lengthening surgery, in which the gracilis and semitendinosus muscles are lengthened to release muscle contractures.

The cells were isolated after biopsy. The following description is of enzymatic digestion of the CP biopsied tissue followed by fluorescent activated cell sorting (FACS). The CP muscle biopsy was placed in a 15 mL container containing 6 mL of digestion buffer (Dulbecco's modified Eagle medium [DMEM; Gibco/Invitrogen, Carlsbad, Calif., USA]; 20 mg/mL collagenase type 1 [Sigma, St. Louis, Mo., USA]; 6 mg/mL dispase II [Roche, Mannheim, Germany]; 6 ku/mL penicillin [Gibco/Invitrogen]; 5 mg/mL streptomycin [Gibco/Invitrogen]). The CP biopsied tissue was transported on ice to a laboratory, and placed in an incubator for 30 minutes, at 5% CO₂ and 37° C. Samples were transferred to a tissue culture plate and dissected using surgical scissors in a sterile tissue culture hood. After the tissue was adequately dissected, it was returned to the incubator for 50 minutes. The plate and the mixture were manually triturated through a 5 mL plastic serological pipette for further mechanical disaggregation of the cells. The resulting cell mixture was incubated for an additional 5 minutes.

Following incubation, the cell mixture was filtered through a 70 μm mesh cell strainer (BD Falcon, San Jose, Calif., USA) into a 50 mL conical-bottom tube. The plate was subsequently washed with 15 mL of DMEM and 10 mL of fetal bovine serum (FBS; Gibco/Invitrogen) and passed through the filter. The final volume was brought to 40 mL using DMEM. The mixture was then centrifuged at 600×g for 10 minutes at 4° C. to obtain a pellet containing mononucleated cells. The cell pellet was re-suspended in a 1 mL fluorescence-activated cell sorting (FACS) buffer (2.5% goat serum; 1 mmol/1 ethylenediaminetetraacetic acid in phosphate-buffered saline [PBS] at pH 7.4).

A 15 μL sample was taken for analysis of total mononuclear cell concentration. An additional 60 μL sample was taken for fluorescence minus one (FMO) controls. Cell-surface markers (see below) were labeled by adding desired conjugated antibodies to the cell suspension. After incubation on ice for 20 minutes, cells were pelleted and washed in 3 mL of FACS buffer. Cells were then washed in 10 mL of FACS buffer and incubated with a secondary antibody on ice for about 20 minutes. Finally, cells were washed in 3 mL of FACS buffer and re-suspended in in 1 mL of FACS buffer for analysis.

Muscle satellite cells were separated from other cell populations in each muscle tissue using the FACSAria II (BD Biosciences, San Jose, Calif., USA). Cells isolated from the CP muscle biopsy were stained for cell surface markers using anti-CD56 antibodies (neural cell adhesion molecule, NCAM, clone HCD56) which reliably identifies myogenic PAX7-positive satellite cells, anti-CD31 antibodies (clone EP3095), which is a marker of endothelial cells, and anti-CD45 antibodies (Abcam ab10559), which is a marker of leukocytes. Using the FACS machine, CD31 and CD45 marker positive cells were combined into a dump channel and discarded, while the remaining CD56-positive cells were collected for cell culture. The resulting satellite cells collected were CD31-negative/CD45-negative/CD56-positive cells due to the removal of endothelial cells and the leukocytes. This muscle cell sorting procedure resulted in the collection of a population of cells committed to undergo myogenesis, rather than neurogenesis, adipogenesis, or other types of cell differentiation.

50,000 satellite cell-derived myoblasts were seeded in each well of 12-well plastic plates coated with gelatin. Isolated and sorted satellite cells from the CP study subject were expanded in vitro in a high serum medium made of Ham's F10 medium (Corning Life Sciences, Tewksbury, Mass.) combined with 20% Fetal Bovine Serum (“FBS”) (Gibco brand, Thermo Fischer Scientific, Waltham, Mass.) 10 mg/ml streptomycin, and 100 units/ml penicillin (Gibco), human recombinant bFGF (5 ng/ml) (Becton Dickenson, Franklin Lakes, N.J.). The satellite cells were frozen in the same high serum medium complemented with 10% DMSO (dimethylsulfoxide) at a concentration of 1-2 million cells/mL.

Cells in the high serum medium were passaged in coated tissue culture dishes. Adhering cell cultures were passaged using a 0.4% trypsin solution to detach cells from the culture dish. Cells were cultured at 37° C. in a 5% CO₂ atmosphere using the high-serum medium. The resulting cell mixture derived from cells from the CP study subject was a myoblast cell preparation.

Myoblasts were grown in the high-serum medium until reaching a 60-70% confluence by visual inspection in 12-well plate culture dishes.

In a first set of wells, the myoblast cell preparations were placed in a high-serum medium until about 70% confluence was reached. They were not exposed to 5-AZA or to reversine. These myoblast cell preparations were then switched to a low-serum medium (as described in Example 1) for four days to promote fusion and myotube formation.

In a second set of wells, the myoblast cell preparations were placed in a high-serum medium until about 70% confluence was reached. Then, the myoblast cell preparations were exposed to 5 μM of 5-azacytidine in the high-serum medium for 6 hours. These myoblast cell preparations were finally switched to a low-serum medium (as described in Example 1) for 4 days to promote fusion and myotube formation.

In a third set of wells, the myoblast cell preparations were placed in a high-serum medium until about 70% confluence was reached. These myoblast cell preparations were then exposed to 5 μM of 5-azacytidine in the high-serum medium for 24 hours. These myoblast cell preparations were finally switched to a low-serum medium (as described in Example 1) for 4 days to promote fusion and myotube formation.

In a fourth set of wells, the myoblast cell preparations were placed in a high-serum medium until about 70% confluence was reached. These myoblast cell preparations were then exposed to 5 μM of reversine (Sigma Aldrich) in the high-serum medium for 3 days. Reversine causes myoblasts to de-differentiate but over time also leads to cell death. Exposing the myoblasts for 3 days reflected a balance between testing the de-differentiation capabilities of reversine as applied to myoblasts while also ensuring that a sufficient quantity of cells would remain for further experimentation at the end period of the exposure. The reversine-treated myoblasts were then switched to a low-serum medium for 4 days to promote fusion and myotube formation.

In a fifth set of wells, the myoblast cell preparations were placed in a high-serum medium until about 70% confluence was reached. These myoblast cell preparations were exposed to 5 μM of reversine and 5 μM of 5-AZA in high-serum medium for 24 hours. These myoblast cell preparations were then exposed to 5 μM of reversine only in high serum medium for an additional 3 days. These same myoblast cell preparations were finally switched to a low-serum medium for 4 days to promote fusion and myotube formation.

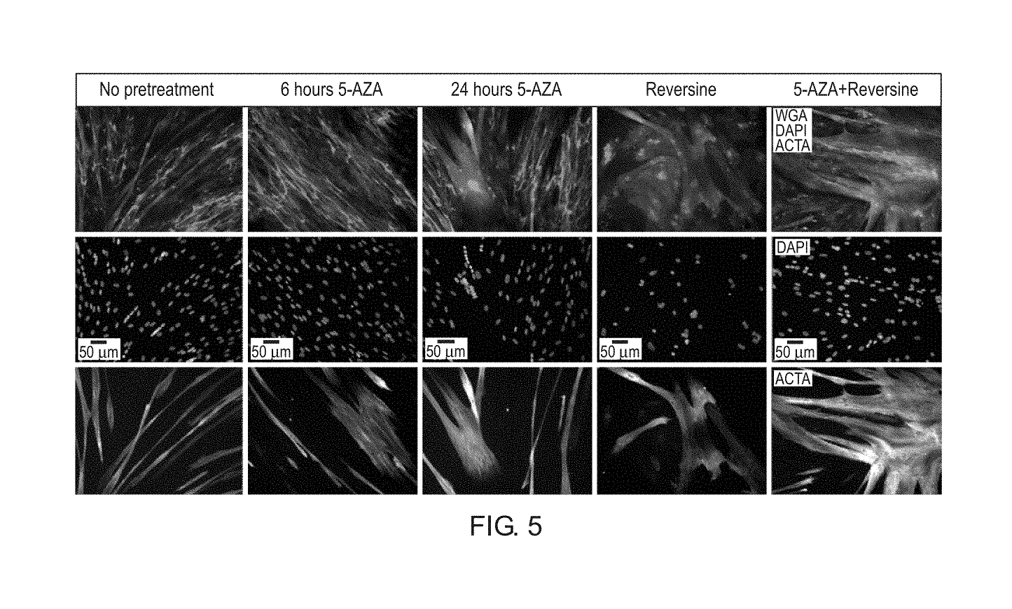

FIG. 5, from left to right, depicts 5 columns with 5 different assays on myotube preparations differentiated from the myoblast cell preparations in the 12-well dishes. The first column reflects myotubes differentiated from a myoblast cell preparation from the first set of wells, which was not exposed to 5-AZA or reversine. The second column reflects myotubes differentiated from a myoblast cell preparation from the second set of wells, which was exposed to 5 μM of 5-AZA in high serum medium for 6 hours. The third column reflects myotubes differentiated from a myoblast cell preparation from the third set of wells, which was exposed to 5 μM of 5-AZA in high serum medium for 24 hours. The fourth column reflects myotubes differentiated from a myoblast cell preparation from the fourth set of wells, which was exposed to 5 μM of reversine in high-serum medium for 3 days. The final, fifth column reflects myotubes differentiated from a myoblast cell preparation from the fifth set of wells, which was exposed to 5 μM reversine and 5 μM of 5-AZA in high serum medium for 24 hours followed by incubating the cells for 3 days with 5 μM reversine alone in high serum medium. The immunostainings on the top row of FIG. 5 identifies lipid membranes (green color, WGA), nuclei (blue color, DAPI) and myotubes (red color, ACTA2). The gray-scale panels (middle row) show staining for nuclei only (DAPI). The gray-scale panels in the bottom row show staining for muscle fibers only (ACTA2).

The effects of combining 5-AZA with reversine on myoblasts prevents cell death and polyploidy. The nuclei in the fourth column are lower in number and suffer from polyploidy, suggesting cell death and genomic aberrations, but the nuclei in the fifth column do not. Reversine also causes morphological changes to the membranes of the cells (shown in the WGA staining), suggesting not only nuclear changes but changes in the shape of the cell as well.

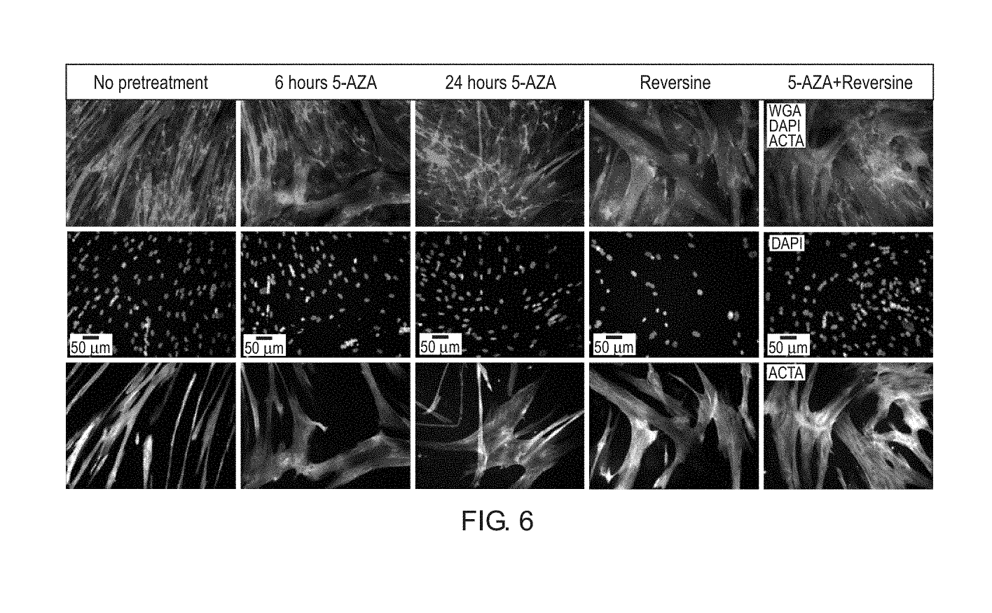

FIG. 6, from left to right, shows assays performed on TD culture preparations that were prepared similarly to those isolated from CP study subjects shown in FIG. 5. Briefly, satellite cells were isolated from muscle biopsies obtained from TD children during surgical procedures, sorted using FACS, plated in gelatin coated dishes, and grown into proliferation medium in 12-well plates. Both FIG. 5 and FIG. 6 morphologically show that co-treatment of satellite cell-derived myoblasts with the cytidine analog, 5-AZA, in combination with the Aurora kinase inhibitor, reversine, yields the most favorable myoblast fusion and myotube formation. The control wherein myoblasts were exposed to reversine alone were shown to promote myoblast fusion and myotube formation, but reversine administration alone led to significant morphological changes to ACTA2-negative cells and nuclei in general (a lower number of oversized cells ACTA2-negative cells, and larger, sometimes polyploid nuclei per field of view; fourth column from the left in FIG. 6). These morphological changes observed in the reversine only treated cells were absent when the myoblast cell preparations were co-treated with reversine and 5-AZA (fifth column from the left) (N=2/group).

The Examples demonstrate that myoblasts derived from satellite cells isolated from contractured muscle in CP patients have an intrinsic impairment to fuse and to produce myotubes in vitro. The demonstrated impairment in the CP-derived myoblast cells appears associated with downregulation of the muscle-specific integrin signaling pathway, which is a critical intracellular pathway that regulates gene expression and leads to myoblast fusion during differentiation. As demonstrated here, the decreased expression of the integrin beta 1D (ITGB1D) protein isoform during differentiation is linked to increased DNA methylation levels on the promoter region of the ITGB1 gene. Together these data also demonstrate that downregulation of integrin ITGB1D-FAK signaling pathway and decreased myogenic potential of differentiating myoblasts may be dependent on small or large-scale changes in DNA methylation patterns affecting genes and protein expression, and activation of pro-myogenic signaling pathways in these cells.

The DNA methyltransferase inhibitor 5-azacytidine, a demethylating agent, restored ITGB1D levels and promoted myogenesis in CP cultures. Example 4 as illustrated in FIGS. 5 and 6 demonstrate that co-treatment of CP patient satellite cells-derived myoblasts with a DNA methyltransferase inhibitor like 5-azacytidine, in combination with an Aurora kinase inhibitor, such as reversine, yields a favorable outcome in terms of myogenesis and rescuing the myogenic potential of CP satellite cells.

### Example 5

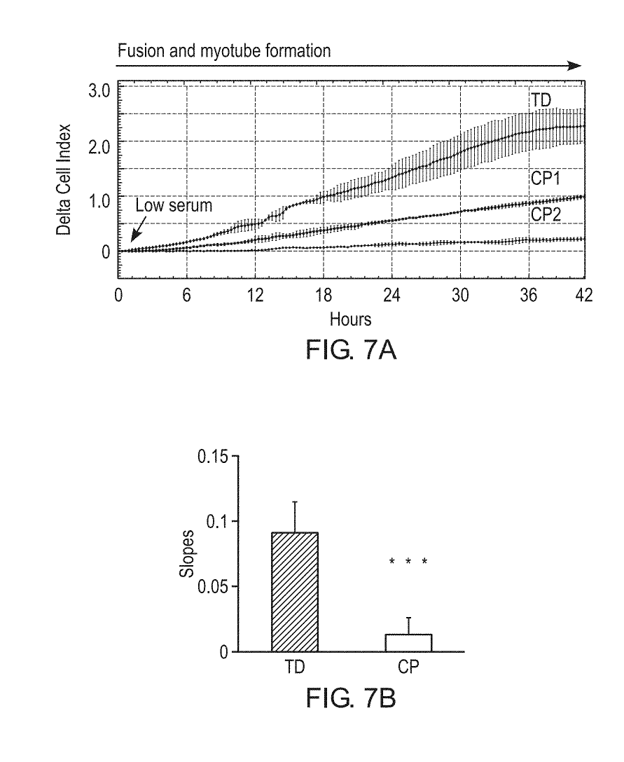

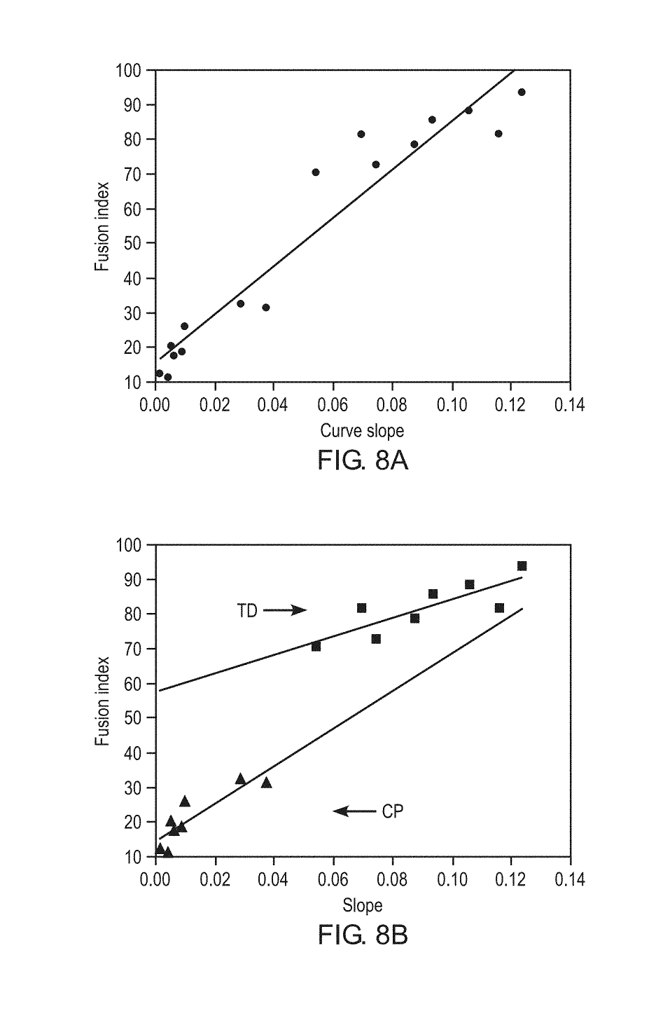

This experiment was done to determine whether the quantity of cell differentiation could be approximated using automated and scalable methods instead of visual inspection and measurement of fusion indices on immunostained cell cultures. Myoblast fusion and myotube formation were examined in vitro using the iCELLigence RTCA Instrument (ACEA Biosciences, San Diego, Calif.) which measures the electric impedance of cells during growth without labeling. In the assays, SC-derived myoblasts were grown in modified 0.5% gelatin-coated 8-well plates with microelectrodes on the bottom of each well for impedance-based detection. 20,000 myoblasts were seeded per well (surface area 0.64 cm²), covering 60-70% of well surface after settling for 12 hours. Cells were cultured at 37° C. in a 5% CO₂ atmosphere using a high-serum medium containing Ham's F10 media (Corning) with 20% Fetal Bovine Serum (FBS, from Gibco), 10 mg/ml streptomycin, 100 units/ml penicillin (Gibco), and human recombinant bFGF (bovine fibroblast growth factor, 5 ng/ml) (BD) until the cells were ˜90% confluent. Differentiation was promoted by switching from high-serum to a low-serum DMEM medium (Gibco) containing 2% Horse Serum (HS, Gibco), 4.5 g/L D-Glucose, L-Glutamine, 10 mg/ml streptomycin, 100 units/ml penicillin (Gibco), and 1 μL/mL insulin solution (Sigma-Aldrich). Myotube formation was associated with a significant increase in cell impedance, measured as a baseline Cell Index (CI) increasing over time. For myotube formation quantification, CI was monitored every 15 minutes for up to 42 hours after the switch to low-serum medium. Two to three replicates of each culture were run at the same time and averaged. Data were analyzed using the RTCA software from ACEA. After normalization and measurement of a Delta Cell Index (DCI), the slope of the curve generated, starting from the time of medium switch to the time when the assay was stopped, was used to quantify rates of fusion and myotube formation over time in CP vs TD samples. FIG. 7A depicts a graph which plots a Delta Cell Index for typically developing cells (TD) and cerebral palsy cells (CP1, CP2). FIG. 7B depicts a chart which compares the average slopes of the plots in FIG. 7A for TD and CP cells. To validate this assay, myoblast fusion index measurements and immunohistochemistry (see below) were performed in parallel. Linear regression quantified the correlation between iCELLigence-generated slope and fusion indices (see FIG. 8).

### Example 6

To investigate whether additional signaling pathways and genes were affected by 5-AZA treatment, real-time qPCR was used to analyze the expression of 91 genes involved in myogenesis, neurogenesis and adipogenesis in fusing CP myoblasts after 24 hours of pretreatment with 5 μM of 5-AZA (Supp. Table 2A). We found that 43 genes were significantly upregulated and 7 genes were significantly downregulated in CP myoblast cultures pretreated with 5-AZA compared to non-treated CP myoblast cultures after 48-72 hours of differentiation (Supp. Table 2B). Several candidate genes that were found to be initially downregulated in differentiating CP cultures when compared to TD (Supp. Table 1B), showed a pattern of upregulation after 5-AZA treatment in CP, including ACTA1, BMI1, CAV3, DES, FOXO1, ITGA7, ITGB1, MEF2A, MEF2C, MET, MYF5, MYF6, NOTCH2, PAK1, PAX3, PPARA, PPARD, SHH, and SIX1. Also, 5-AZA pretreatment led to upregulation of transcription regulators involved in myogenesis and other signaling pathways (BMI1, SIX1, SIX4, EGR3, FOXO1, MAPK14, MEF2A, MEF2C, MYF5, MYOG, NEUROD2, NOTCH2, PAX3, PAX7, PPARA, PPARD, PPARG, RPBJ, RARA). Specific signaling pathways were also upregulated, including NOTCH (DLL1, JAG1, JAG2, NOTCH2, RPBJ) and the integrin and focal adhesion pathways (BCL2, MET, ACTN2, CAV3, ITGA7, ITGB1, PAK1). Interestingly, upregulation of myogenic factors was also associated with upregulation of factors potentially involved in regulatory networks of other differentiation programs, including neurogenesis and adipogenesis (NEUROD2, PPARG). Together these data suggest that downregulation of the integrin ITGB1D-FAK signaling pathway and decreased myogenic potential of differentiating myoblasts may be dependent on small or large-scale changes in DNA methylation patterns affecting gene expression and activation of pro-myogenic signaling pathways in these cells. Impairment in other signaling pathways involved in myoblast fusion and myotube formation (e.g., TMEM8C/Myomaker, Rho GTPases, MAPKs, Calcineurin-NFATc2, NF-KB, WNT, MMPs) may also contribute to the decrease in myogenic potential of CP myoblasts.

Myoblasts, treated or non-treated with 5 μM 5-AZA for 24 hours, were cultured in high serum medium until they reached 80-90% confluence. After switch to low-serum medium, myoblast preparations were allowed to differentiate for 24-72 hours before RNA extraction. Total RNA was isolated and purified from myoblast cell culture preparations using Trizol reagent (Ambion—Life Technologies) according to manufacturer's recommendations. First-strand cDNA synthesis from purified RNAs and qPCR amplification was performed using the GoTaq 2-Step RT-qPCR System kit (Promega) according to manufacturer's protocols. Resulting cDNAs were subjected to SYBR Green-based real-time amplification using a C1000 Thermal Cycler apparatus (Bio-Rad). Each PCR amplification reaction (20 μl volume) contained 100 ng of starting cDNA template and primers amplifying a specific gene. Gene expression profiling during myoblast differentiation was investigated using predesigned 96-well gene panels for use with SYBR Green (SABioscience) (Tables 1A and 2A).

Table 1B below indicates genes, which were inhibited or upregulated in non-treated CP cells as compared to non-treated TD cells. The genes provided in the 96-well panels are listed in Table 1A. In Table 1B, inhibition is indicated by a negative value in the Regulation column and upregulation is indicated by a positive value in the Regulation column. For example, the ITGB1 gene was inhibited in CP cells 2.4 times as much as the ITGB1 gene in TD cells.

Table 2B below indicates genes which were inhibited or upregulated in treated CP cells as compared to non-treated TD cells. The genes provided in the 96-well panels are listed in Table 2A. In Table 2B, inhibition is indicated by a negative value in the Regulation column and upregulation is indicated by a positive value in the Regulation column. For example, the ITGB1 gene was upregulated in treated CP cells 2.1 times as compared to the ITGB1 gene in non-treated CP cells.

The predesigned 96-well gene panels utilized expertly designed and experimentally validated PCR primers and assays and included 3 reference genes for relative gene expression normalization and quantification: HPRT1, TBP and AP3DI. These reference genes were specifically validated for our experimental treatment and conditions and were selected among 15 experimentally tested candidates and analyzed using the software packages Bestkeeper, geNorm and Normfinder. Cycling conditions for SYBR Green primers consisted of an initial step at 50° C. (2 minutes) and a first denaturizing step at 95° C. (2 minutes), followed by 50 cycles of a thermal step protocol consisting of 95° C. (20 seconds), 60° C. (20 seconds) and 72° C. (20 seconds). A standard melt curve profiling consisting of a 65-95° C. thermal ramp was performed at the end of each protocol. Gene expression quantification was performed using Bio-Rad CFX Manager software. Positively and negatively regulated genes were selected following a P<0.05 and a 2 or 0.5 expression level respectively. Functional gene classification was performed using The Database for Annotation, Visualization and Integrated Discovery (DAVID) v6.8 (at <https://david.ncifcrf.gov/>).

TABLE 1A

| | | Gene |
|---|---|---|
| Well | Gene Name | Symbol |
| A1 | actin, alpha 1, skeletal muscle | ACTA1 |
| A2 | ATPase, Ca++ transporting, cardiac muscle, fast twitch 1 | ATP2A1 |
| A3 | calpastatin | CAST |
| A4 | dystrophin | DMD |
| A5 | histone deacetylase 5 | HDAC5 |
| A6 | interleukin 6 (interferon, beta 2) | IL6 |
| A7 | myocyte enhancer factor 2C | MEF2C |
| A8 | myogenic differentiation 1 | MYOD1 |
| A9 | pyruvate dehydrogenase kinase, isozyme 4 | PDK4 |
| A10 | ras homolog gene family, member A | RHOA |
| A11 | troponin I type 2 (skeletal, fast) | TNNI2 |
| A12 | hypoxanthine phosphoribosyltransferase 1 | HPRT1 |
| B1 | actin, beta | ACTB |
| B2 | beta-2-microglobulin | B2M |
| B3 | caveolin 1, caveolae protein, 22 kDa | CAV1 |
| B4 | dystrophia myotonica-protein kinase | DMPK |
| B5 | hexokinase 2 | HK2 |
| B6 | leptin | LEP |
| B7 | matrix metallopeptidase 9 (gelatinase B, 92 kDa gelatinase, 92 kDa type IV | MMP9 |
| | collagenase) | |
| B8 | myogenin (myogenic factor 4) | MYOG |
| B9 | peroxisome proliferator-activated receptor gamma | PPARG |
| B10 | ribosomal protein, large, P0 | RPLP0 |
| B11 | troponin T type 1 (skeletal, slow) | TNNT1 |
| B12 | PrimePCR DNA Contamination Control Assay | gDNA |
| C1 | activin A receptor, type IIB | ACVR2B |
| C2 | B-cell CLL/lymphoma 2 | BCL2 |
| C3 | caveolin 3 | CAV3 |
| C4 | dysferlin, limb girdle muscular dystrophy 2B (autosomal recessive) | DYSF |
| C5 | insulin-like growth factor 1 (somatomedin C) | IGF1 |
| C6 | lamin A/C | LMNA |
| C7 | myostatin | MSTN |
| C8 | myotilin | MYOT |
| C9 | peroxisome proliferator-activated receptor gamma, coactivator 1 alpha | PPARGC1A |
| C10 | ribosomal protein S6 kinase, 70 kDa, polypeptide 1 | RPS6KB1 |
| C11 | troponin T type 3 (skeletal, fast) | TNNT3 |
| C12 | PrimePCR Positive Control Assay | PCR |
| D1 | adiponectin, C1Q and collagen domain containing | ADIPOQ |
| D2 | bone morphogenetic protein 4 | BMP4 |
| D3 | crystallin, alpha B | CRYAB |
| D4 | F-box protein 32 | FBXO32 |
| D5 | insulin-like growth factor 2 (somatomedin A) | IGF2 |
| D6 | mitogen-activated protein kinase 1 | MAPK1 |
| D7 | muscle, skeletal, receptor tyrosine kinase | MUSK |
| D8 | nebulin | NEB |
| D9 | peroxisome proliferator-activated receptor gamma, coactivator 1 beta | PPARGC1B |
| D10 | sarcoglycan, alpha (50 kDa dystrophin-associated glycoprotein) | SGCA |
| D11 | tripartite motif containing 63 | TRIM63 |
| D12 | PrimePCR RNA Quality Assay | RQ1 |
| E1 | adrenergic, beta-2-, receptor, surface | ADRB2 |
| E2 | calcium/calmodulin-dependent protein kinase II gamma | CAMK2G |
| E3 | citrate synthase | CS |
| E4 | fibroblast growth factor 2 (basic) | FGF2 |
| E5 | insulin-like growth factor binding protein 3 | IGFBP3 |
| E6 | mitogen-activated protein kinase 14 | MAPK14 |
| E7 | myogenic factor 5 | MYF5 |
| E8 | nuclear factor of kappa light polypeptide gene enhancer in B-cells 1 | NFKB1 |
| E9 | protein phosphatase 3, catalytic subunit, alpha isozyme | PPP3CA |
| E10 | solute carrier family 2 (facilitated glucose transporter), member 4 | SLC2A4 |
| E11 | titin | TTN |
| E12 | TATA box binding protein | TBP |
| F1 | agrin | AGRN |
| F2 | calpain 2, (m/II) large subunit | CAPN2 |
| F3 | catenin (cadherin-associated protein), beta 1, 88 kDa | CTNNB1 |
| F4 | forkhead box O1 | FOXO1 |
| F5 | insulin-like growth factor binding protein 5 | IGFBP5 |
| F6 | mitogen-activated protein kinase 3 | MAPK3 |
| F7 | myogenic factor 6 (herculin) | MYF6 |
| F8 | nitric oxide synthase 2, inducible | NOS2 |
| F9 | protein kinase, AMP-activated, beta 2 non-catalytic subunit | PRKAB2 |
| F10 | transforming growth factor, beta 1 | TGFB1 |
| F11 | utrophin | UTRN |
| F12 | adaptor-related protein complex 3, delta 1 subunit | AP3D1 |
| G1 | v-akt murine thymoma viral oncogene homolog 1 | AKT1 |
| G2 | calpain 3, (p94) | CAPN3 |
| G3 | dystroglycan 1 (dystrophin-associated glycoprotein 1) | DAG1 |
| G4 | forkhead box O3 | FOXO3 |
| G5 | inhibitor of kappa light polypeptide gene enhancer in B-cells, kinase beta | IKBKB |
| G6 | mitogen-activated protein kinase 8 | MAPK8 |
| G7 | myosin, heavy chain 1, skeletal muscle, adult | MYH1 |
| G8 | paired box 3 | PAX3 |
| G9 | protein kinase, AMP-activated, gamma 1 non-catalytic subunit | PRKAG1 |
| G10 | tumor necrosis factor | TNF |
| G11 | TATA box binding protein | TBP |
| G12 | hypoxanthine phosphoribosyltransferase 1 | HPRT1 |
| H1 | v-akt murine thymoma viral oncogene homolog 2 | AKT2 |
| H2 | caspase 3, apoptosis-related cysteine peptidase | CASP3 |
| H3 | desmin | DES |
| H4 | glucuronidase, beta | GUSB |
| H5 | interleukin 1, beta | IL1B |
| H6 | myoglobin | MB |
| H7 | myosin, heavy chain 2, skeletal muscle, adult | MYH2 |
| H8 | paired box 7 | PAX7 |
| H9 | protein kinase, AMP-activated, gamma 3 non-catalytic subunit | PRKAG3 |
| H10 | troponin C type 1 (slow) | TNNC1 |
| H11 | adaptor-related protein complex 3, delta 1 subunit | AP3D1 |
| H12 | ribosomal protein L13a | RPL13A |

TABLE 1B

| Gene | Expression | Regulation | Description |
|---|---|---|---|
| ACTA1 | 0.01 | −75.1 | actin, alpha 1, skeletal muscle |
| MYF6 | 0.02 | −40.1 | myogenic factor 6 |
| BMI1 | 0.03 | −30.1 | BMI1 proto-oncogene, polycomb |
| | | | ring finger |
| NGFR | 0.04 | −23.9 | nerve growth factor receptor |
| MEF2C | 0.06 | −17.3 | myocyte enhancer factor 2C |
| MEF2A | 0.07 | −14.4 | myocyte enhancer factor 2A |
| RB1 | 0.07 | −13.5 | RB transcriptional corepressor 1 |
| SMAD1 | 0.08 | −13.3 | SMAD family member 1 |
| ITGAV | 0.09 | −11.6 | integrin subunit alpha V |
| CBFB | 0.09 | −11.6 | core-binding factor beta subunit |
| PTEN | 0.09 | −11.0 | phosphatase and tensin homolog |
| MAPK14 | 0.09 | −11.0 | mitogen-activated protein kinase |
| | | | 14 |
| F2R | 0.12 | −8.2 | coagulation factor II thrombin |
| | | | receptor |
| FOXO1 | 0.13 | −7.6 | forkhead box O1 |
| GSK3B | 0.13 | −7.6 | glycogen synthase kinase 3 beta |
| TNNI3 | 0.14 | −7.2 | troponin I3, cardiac type |
| NR4A1 | 0.14 | −7.1 | nuclear receptor subfamily 4 group |
| | | | A member 1 |
| PPARD | 0.15 | −6.5 | peroxisome proliferator activated |
| | | | receptor delta |
| SIX1 | 0.16 | −6.3 | SIX homeobox 1 |
| HDAC1 | 0.16 | −6.2 | histone deacetylase 1 |
| CDK5 | 0.16 | −6.2 | cyclin dependent kinase 5 |
| EWSR1 | 0.17 | −6.0 | EWS RNA binding protein 1 |
| PPARA | 0.18 | −5.7 | peroxisome proliferator activated |
| | | | receptor alpha |
| SHH | 0.18 | −5.6 | sonic hedgehog |
| TBP | 0.18 | −5.4 | TATA-box binding protein |
| CTNNB1 | 0.19 | −5.3 | catenin beta 1 |
| CAV3 | 0.22 | −4.6 | caveolin 3 |
| BMP4 | 0.23 | −4.3 | bone morphogenetic protein 4 |
| TPM1 | 0.24 | −4.2 | tropomyosin 1 (alpha) |
| MYF5 | 0.26 | −3.8 | myogenic factor 5 |
| NOTCH2 | 0.29 | −3.5 | notch 2 |
| DES | 0.29 | −3.4 | desmin |
| ITGA7 | 0.31 | −3.2 | integrin subunit alpha 7 |
| LMNA | 0.34 | −2.9 | lamin A/C |
| MET | 0.37 | −2.7 | MET proto-oncogene, receptor |
| | | | tyrosine kinase |
| GSK3A | 0.38 | −2.6 | glycogen synthase kinase 3 alpha |
| PAK1 | 0.38 | −2.6 | p21 (RAC1) activated kinase 1 |
| ACTA2 | 0.39 | −2.6 | actin, alpha 2, smooth muscle, |
| | | | aorta |
| PAX3 | 0.41 | −2.4 | paired box 3 |
| ITGB1 | 0.42 | −2.4 | integrin subunit beta 1 |
| MYLK | 0.46 | −2.2 | myosin light chain kinase |
| CAV1 | 0.47 | −2.1 | caveolin 1 |
| MYH9 | 26.31 | 26.3 | myosin heavy chain 9 |
| PDGFRB | 15.64 | 15.6 | platelet derived growth factor |
| | | | receptor beta |
| RARA | 14.82 | 14.8 | retinoic acid receptor alpha |
| TNC | 10.69 | 10.7 | tenascin C |
| NRG1 | 8.28 | 8.3 | neuregulin 1 |
| ACTG1 | 5.60 | 5.6 | actin gamma 1 |
| FHL2 | 4.29 | 4.3 | four and a half LIM domains 2 |
| PDGFRA | 3.14 | 3.1 | platelet derived growth factor |
| | | | receptor alpha |
| MYOG | 3.06 | 3.1 | myogenin |

TABLE 2A

| Well | Gene Name | Gene Symbol |
|---|---|---|
| A1 | actin, alpha 1, skeletal muscle | ACTA1 |
| A2 | actin, alpha 2, smooth muscle, aorta | ACTA2 |
| A3 | actin, beta | ACTB |
| A4 | actin, gamma 1 | ACTG1 |
| A5 | actinin, alpha 2 | ACTN2 |
| A6 | ADAM metallopeptidase domain 12 | ADAM12 |
| A7 | ATPase, Ca++ transporting, cardiac muscle, fast twitch 1 | ATP2A1 |
| A8 | ATPase, Ca++ transporting, cardiac muscle, slow twitch 2 | ATP2A2 |
| A9 | BCL2-associated X protein | BAX |
| A10 | B-cell CLL/lymphoma 2 | BCL2 |
| A11 | BMI1 polycomb ring finger oncogene | BMI1 |
| A12 | bone morphogenetic protein 2 | BMP2 |
| B1 | bone morphogenetic protein 4 | BMP4 |
| B2 | caveolin 1, caveolae protein, 22 kDa | CAV1 |
| B3 | caveolin 3 | CAV3 |
| B4 | core-binding factor, beta subunit | CBFB |
| B5 | cyclin-dependent kinase 5 | CDK5 |
| B6 | cyclin-dependent kinase inhibitor 1A (p21, Cip1) | CDKN1A |
| B7 | catenin (cadherin-associated protein), beta 1, 88 kDa | CTNNB1 |
| B8 | desmin | DES |
| B9 | delta-like 1 homolog (Drosophila) | DLK1 |
| B10 | delta-like 1 (Drosophila) | DLL1 |
| B11 | dystrophin | DMD |
| B12 | early growth response 3 | EGR3 |
| C1 | four and a half LIM domains 1 | FHL1 |
| C2 | four and a half LIM domains 2 | FHL2 |
| C3 | forkhead box O1 | FOXO1 |
| C4 | glycogen synthase kinase 3 alpha | GSK3A |
| C5 | glycogen synthase kinase 3 beta | GSK3B |
| C6 | histone deacetylase 1 | HDAC1 |
| C7 | histone deacetylase 2 | HDAC2 |
| C8 | integrin, alpha 4 (antigen CD49D, alpha 4 subunit of VLA-4 | ITGA4 |
| | receptor) | |
| C9 | integrin, alpha 7 | ITGA7 |
| C10 | integrin, alpha 9 | ITGA9 |
| C11 | integrin, alpha V (vitronectin receptor, alpha polypeptide, | ITGAV |
| | antigen CD51) | |
| C12 | integrin, beta 1 (fibronectin receptor, beta polypeptide, antigen | ITGB1 |
| | CD29 includes MDF2, MSK12) | |
| D1 | jagged 1 | JAG1 |
| D2 | jagged 2 | JAG2 |
| D3 | lamin A/C | LMNA |
| D4 | low density lipoprotein receptor-related protein 4 | LRP4 |
| D5 | mitogen-activated protein kinase 14 | MAPK14 |
| D6 | myocyte enhancer factor 2A | MEF2A |
| D7 | myocyte enhancer factor 2C | MEF2C |
| D8 | met proto-oncogene (hepatocyte growth factor receptor) | MET |
| D9 | myogenic factor 5 | MYF5 |
| D10 | myogenic factor 6 (herculin) | MYF6 |
| D11 | myosin, heavy chain 1, skeletal muscle, adult | MYH1 |
| D12 | myosin, heavy chain 2, skeletal muscle, adult | MYH2 |
| E1 | myosin, heavy chain 3, skeletal muscle, embryonic | MYH3 |
| E2 | myosin, heavy chain 4, skeletal muscle | MYH4 |
| E3 | myosin, heavy chain 6, cardiac muscle, alpha | MYH6 |
| E4 | myosin, heavy chain 7, cardiac muscle, beta | MYH7 |
| E5 | myosin, heavy chain 8, skeletal muscle, perinatal | MYH8 |
| E6 | myosin, heavy chain 9, non-muscle | MYH9 |
| E7 | myogenic differentiation 1 | MYOD1 |
| E8 | myogenin (myogenic factor 4) | MYOG |
| E9 | neural cell adhesion molecule 1 | NCAM1 |
| E10 | neurogenic differentiation 2 | NEUROD2 |
| E11 | nerve growth factor receptor | NGFR |
| E12 | notch 1 | NOTCH1 |
| F1 | notch 2 | NOTCH2 |
| F2 | neuregulin 1 | NRG1 |
| F3 | p21 protein (Cdc42/Rac)-activated kinase 1 | PAK1 |
| F4 | paired box 3 | PAX3 |
| F5 | paired box 7 | PAX7 |
| F6 | proliferating cell nuclear antigen | PCNA |
| F7 | platelet-derived growth factor receptor, alpha polypeptide | PDGFRA |
| F8 | platelet-derived growth factor receptor, beta polypeptide | PDGFRB |
| F9 | peroxisome proliferator-activated receptor alpha | PPARA |
| F10 | peroxisome proliferator-activated receptor delta | PPARD |
| F11 | peroxisome proliferator-activated receptor gamma | PPARG |
| F12 | phosphatase and tensin homolog | PTEN |
| G1 | retinoic acid receptor, alpha | RARA |
| G2 | retinoblastoma 1 | RB1 |
| G3 | RNA binding protein, fox-1 homolog (C. elegans) 2 | RBFOX2 |
| G4 | recombination signal binding protein for immunoglobulin kappa | RBPJ |
| | J region | |
| G5 | ras homolog gene family, member A | RHOA |
| G6 | sonic hedgehog | SHH |
| G7 | SIX homeobox 1 | SIX1 |
| G8 | SIX homeobox 4 | SIX4 |
| G9 | SMAD family member 1 | SMAD1 |
| G10 | serum response factor (c-fos serum response element-binding | SRF |
| | transcription factor) | |
| G11 | transmembrane protein 8C | TMEM8C |
| G12 | tenascin C | TNC |
| H1 | troponin I type 3 (cardiac) | TNNI3 |
| H2 | tropomyosin 1 (alpha) | TPM1 |
| H3 | twist homolog 2 (Drosophila) | TWIST2 |
| H4 | vinculin | VCL |
| H5 | wingless-type MMTV integration site family, member 1 | WNT1 |
| H6 | wingless-type MMTV integration site family, member 7A | WNT7A |
| H7 | hydroxymethylbilane synthase | HMBS |
| H8 | hypoxanthine phosphoribosyltransferase 1 | HPRT1 |
| H9 | TATA box binding protein | TBP |
| H10 | adaptor-related protein complex 3, delta 1 subunit | AP3D1 |
| H11 | ribosomal protein L13a | RPL13A |
| H12 | ribosomal protein L13a | RPL13A |

TABLE 2B

| Gene | Expression | Regulation | Description |
|---|---|---|---|
| NEUROD2 | 56.33 | 56.3 | neuronal differentiation 2 |
| PPARG | 24.42 | 24.4 | peroxisome proliferator activated receptor |
| | | | gamma |
| SHH | 23.75 | 23.8 | sonic hedgehog |
| DLK1 | 16.51 | 16.5 | delta like non-canonical Notch ligand 1 |
| EGR3 | 14.93 | 14.9 | early growth response 3 |
| PAX3 | 12.50 | 12.5 | paired box 3 |
| JAG2 | 10.19 | 10.2 | jagged 2 |
| MEF2A | 10.08 | 10.1 | myocyte enhancer factor 2A |
| ATP2A1 | 7.87 | 7.9 | ATPase sarcoplasmic/endoplasmic reticulum |
| | | | Ca²⁺ transporting 1 |
| MYOG | 7.72 | 7.7 | myogenin |
| FOXO1 | 7.30 | 7.3 | forkhead box O1 |
| DLL1 | 7.05 | 7.0 | delta like canonical Notch ligand 1 |
| MYH2 | 6.99 | 7.0 | myosin heavy chain 2 |
| MYF5 | 6.99 | 7.0 | myogenic factor 5 |
| MYH3 | 6.74 | 6.7 | myosin heavy chain 3 |
| NCAM1 | 6.55 | 6.6 | neural cell adhesion molecule 1 |
| DES | 6.23 | 6.2 | desmin |
| ACTN2 | 5.96 | 6.0 | actinin alpha 2 |
| MEF2C | 5.79 | 5.8 | myocyte enhancer factor 2C |
| BCL2 | 5.33 | 5.3 | BCL2, apoptosis regulator |
| ACTA1 | 5.23 | 5.2 | actin, alpha 1, skeletal muscle |
| PAK1 | 4.49 | 4.5 | p21 (RAC1) activated kinase 1 |
| ITGA7 | 4.22 | 4.2 | integrin subunit alpha 7 |
| RBPJ | 3.84 | 3.8 | recombination signal binding protein for |
| | | | immunoglobulin kappa J region |
| MYF6 | 3.77 | 3.8 | myogenic factor 6 |
| TMEM8C | 3.59 | 3.6 | transmembrane protein 8C |
| PPARD | 3.55 | 3.6 | peroxisome proliferator activated receptor delta |
| ATP2A2 | 3.26 | 3.3 | ATPase sarcoplasmic/endoplasmic reticulum |
| | | | Ca²⁺ transporting 2 |
| SIX4 | 3.20 | 3.2 | SIX homeobox 4 |
| NOTCH2 | 3.12 | 3.1 | notch 2 |
| LRP4 | 3.01 | 3.0 | LDL receptor related protein 4 |
| BMI1 | 2.97 | 3.0 | BMI1 proto-oncogene, polycomb ring finger |
| CAV3 | 2.92 | 2.9 | caveolin 3 |
| PAX7 | 2.88 | 2.9 | paired box 7 |
| MAPK14 | 2.84 | 2.8 | mitogen-activated protein kinase 14 |
| PPARA | 2.69 | 2.7 | peroxisome proliferator activated receptor alpha |
| JAG1 | 2.61 | 2.6 | jagged 1 |
| MYH8 | 2.51 | 2.5 | myosin heavy chain 8 |
| SIX1 | 2.21 | 2.2 | SIX homeobox 1 |
| RARA | 2.19 | 2.2 | retinoic acid receptor alpha |
| MYH7 | 2.15 | 2.1 | myosin heavy chain 7 |
| ITGB1 | 2.11 | 2.1 | integrin subunit beta 1 |
| MET | 2.07 | 2.1 | MET proto-oncogene, receptor tyrosine kinase |
| ACTG1 | 0.09 | −10.7 | actin gamma 1 |
| MYH9 | 0.17 | −5.8 | myosin heavy chain 9 |
| CDKN1A | 0.23 | −4.4 | cyclin dependent kinase inhibitor 1A |
| PDGFRB | 0.34 | −2.9 | platelet derived growth factor receptor beta |
| PDGFRA | 0.38 | −2.6 | platelet derived growth factor receptor alpha |
| FHL2 | 0.45 | −2.2 | four and a half LIM domains 2 |
| NRG1 | 0.47 | −2.1 | neuregulin 1 |

### Example 7

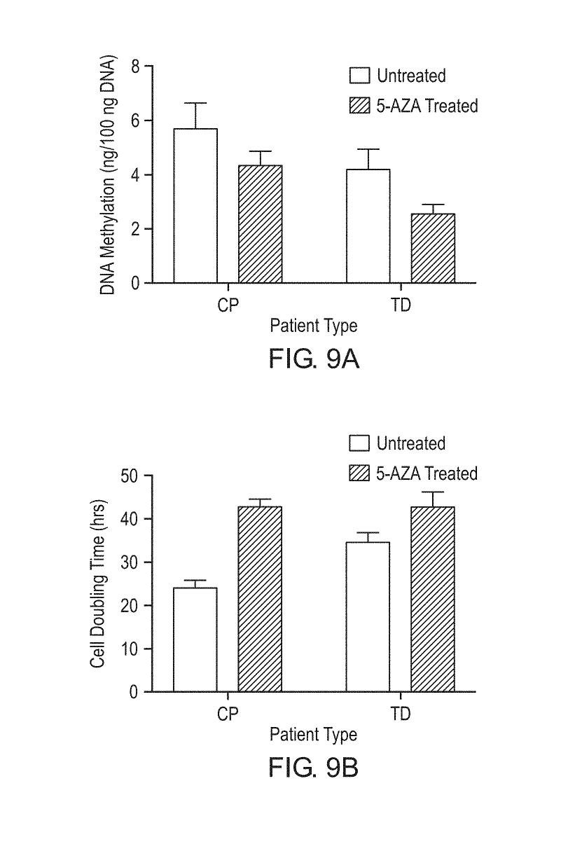

In FIG. 9A, global methylation levels of DNA were investigated in proliferating CP and TD myoblast cultures that were treated or non-treated with 5-AZA. Methylation levels were measured using a MethylFlash Methylated DNA Quantification Kit (EpiGentek, P-1034-96). Myoblasts were seeded into gelatin-coated 10 cm plates. Myoblast cell preparations from CP and TD study subjects were exposed to 5 μM of 5-AZA in proliferation medium. 5-AZA was introduced while the myoblast cell preparations were being developed in the high-serum medium, once the myoblast cell preparations were at about 60% of cell plate confluence by visual inspection. The myoblast cell preparations each were cultured in a high-serum medium in the presence of 5 μM of 5-AZA for 24 hours as described earlier. The myoblast cell preparations then had the 5-AZA washed off by switching the myoblast cell preparations each to fresh high-serum medium, and cultured to further expand to 90% of confluence. DNA was extracted from these cultures using the “DNA Purification from Cultured Cells” protocol as described in the Gentra Puregene DNA isolation kit (Qiagen, 158388). FIG. 9A illustrates that 5-AZA treatment caused global DNA hypomethylation in both CP and TD patients.

A cell impedance-measuring xCELLigence RTCA DP instrument was used to quantify rates of “cell doubling time” (a measurement of cell proliferation) in CP and TD myoblast preparations, treated and non-treated with 5-AZA, as illustrated in FIG. 9B. Proliferating myoblasts were treated with 5 μM 5-AZA (Sigma-Aldrich) for 24 hours to induce DNA hypomethylation. Other cell preparations were not treated with 5-AZA to serve as a control. Following treatment, myoblasts were harvested from exponential growth phase cultures by a standardized detachment procedure using 0.05% Trypsin-EDTA and counted. Myoblast suspensions were seeded into wells of gelatin-coated electrode array plates (E-plates) at 400 and 800 cells/well. Two replicates of each cell concentration were used in each test. Plates were locked in the RTCA DP device in the incubator and the impedance value of each well was automatically monitored by the xCELLigence system and expressed as a Cell Index value (CI). CP and TD myoblasts were incubated during 10 days in a high-serum medium containing Ham's FIO media (Corning) with 20% Fetal Bovine Serum (FBS, from Gibco), 10 mg/ml streptomycin, 100 units/ml penicillin (Gibco), and human recombinant bFGF (bovine fibroblast growth factor, 5 ng/ml) (BD). CI was recorded every 15 minutes for the entirety of the period. Cell doubling time was calculated from the exponential phase of the growth curve generated by the CI values using the supplied RTCA software. Untreated CP myoblasts exhibited a faster cell doubling time compared to TD myoblasts. Unexpectedly, treatment with 5-AZA resulted in similar myoblast cell doubling times between CP and TD myoblasts.

1. A method for culturing muscle cells isolated from a contractured muscle of a patient comprising:
isolating cells from contractured muscle; and
culturing the isolated muscle cells in a proliferation medium containing a DNA methyltransferase inhibitor to obtain cultured cells from contractured muscle.

2. The method of claim 1, wherein the DNA methyltransferase inhibitor is a cytidine analog including any salts, hydrates, solvates, prodrugs, and any crystal forms in which they may occur.

3. The method of claim 1 wherein the DNA methyltransferase inhibitor is selected from the group consisting of: 5-azacytidine or a non-nucleoside inhibitor of DNA methyltransferase, caffeic acid, chlorogenic acid, (−)-epigallocatechin gallate, hydralazine, procaine, N-phthalyl-L-tryptophan (RG108), and any salts, hydrates, solvates, prodrugs, and crystal forms in which they may occur of the DNA methyltransferase inhibitor.

4. The method of claim 1, wherein the culturing step is for a period of 1 to 24 hours.

5. The method of claim 1, further comprising culturing the cultured cells from contractured muscle in a proliferation medium containing an Aurora kinase inhibitor.

6. The method of claim 1, further comprising:
removing the DNA methyltransferase inhibitor from the cultured cells from contractured muscle; and
culturing the cultured cells from contractured muscle in a second proliferation medium containing an Aurora kinase inhibitor.

7. The method of claim 5, wherein the culturing of the cultured cells from contractured muscle in the proliferation media having the Aurora kinase inhibitor occurs after culturing the cells isolated from contractured muscle in the proliferation medium having the DNA methyltransferase inhibitor.

8. The method of claim 5, wherein the Aurora kinase inhibitor is 2-(4-morpholinoanilino)-N6-cyclohexyladenine or an analog thereof.

9. The method of claim 1, wherein the culturing is for about at least 6 hours.

10. The method of claim 1, wherein the cells isolated from contractured muscle comprise muscle satellite cells or myoblasts.

11. The method of claim 10, wherein the myoblasts are derived in vitro from muscle satellite cells.

12. The method of claim 1, wherein the cells isolated from contractured muscle comprise a portion of muscle fiber.

13. The method of claim 12, wherein the portion of muscle fiber is dissected or cultured in vitro.

14. The method of claim 10, wherein the myoblasts are derived from a muscle fiber.

15. The method of claim 14, wherein the muscle fiber is a dissected muscle fiber or cultured in vitro.

16. The method of claim 1, wherein the cells isolated from contractured muscle are obtained from a patient having cerebral palsy.

17. The method of claim 1, wherein the culturing in the presence of the DNA methyltransferase inhibitor is for about 1 to about 24 hours, and the method further comprises administering an Aurora kinase inhibitor to the cultured cells from contractured muscle for up to four days.

18. The method of claim 17, wherein the DNA methyltransferase inhibitor is 5-azacytidine and the Aurora kinase inhibitor is 2-(4-morpholinoanilino)-N6-cyclohexyladenine.

19. The method of claim 17, wherein the Aurora kinase inhibitor is administered at about the same time as the DNA methyltransferase inhibitor.

20. Isolated cells from contractured muscle obtained from a patient, wherein the isolated cells from contractured muscle have been cultured in a proliferation medium in vitro in the presence of a DNA methyltransferase inhibitor.

21. The isolated cells from contractured muscle of claim 20, wherein the isolated cells from contractured muscle have also been cultured in the presence of an Aurora kinase inhibitor.

22. The isolated cells from contractured muscle of claim 20 or 21, wherein the isolated cells from contractured muscle are differentiated into myoblasts prior to culturing in the proliferation medium.

23. The isolated cells from contractured muscle of claim 20, wherein the isolated cells from contractured muscle are satellite cells.

24. The isolated cells from contractured muscle of claim 21, wherein the isolated cells from contractured muscle have been cultured in the concurrent presence of the DNA methyltransferase inhibitor and the Aurora kinase inhibitor.

25. The isolated cells from contractured muscle of claim 21, wherein the isolated cells from contractured muscle have been cultured in the presence of the DNA methyltransferase inhibitor for about 1 to about 24 hours and have been cultured in the presence of the Aurora kinase inhibitor for up to four days.

26. The isolated cells from contractured muscle of claim 20, wherein the isolated cells from contractured muscle are passaged 1 to 6 times before culturing in the presence of the DNA methyltransferase inhibitor.

27. The isolated cells from contractured muscle of claim 20, wherein the isolated cells from contractured muscle are cryopreserved before the culturing of the isolated cells from contractured muscle in vitro in the presence of the DNA methyltransferase inhibitor.

28. An isolated muscle cell from contractured muscle obtained by the method of claim 2.

29. A method for treating a patient having cerebral palsy comprising the step of administering to the patient the isolated cells from contractured muscle obtained by the method of claim 20.

30. A method of treating skeletal muscle contractures, skeletal muscle atrophy, or remodeling muscle tissue in a patient in need thereof comprising: administering the isolated cells from contractured muscle obtained by the method of claim 20, wherein the cells are administered intramuscularly at least once.

31. A method of treating skeletal muscle contractures, skeletal muscle atrophy or remodeling muscle tissue in a patient in need thereof comprising: administering the isolated cells from contractured muscle obtained by the method of claim 20, wherein the cells are administered by surgical transplant at least once.

32. The method of claim 1, wherein the step of culturing the isolated muscle cells in the proliferation medium containing a DNA methyltransferase inhibitor comprises upregulating ITGB1 gene in the cells.

33. The method of claim 1, wherein the DNA methyltransferase inhibitor is present in the amount of about 0.1 to about 100 μM.


SEQUENCE LISTING (6 sequences)

SEQ ID NO: 1 - DNA, 20 bp, Artificial Sequence
  Chemically synthesized; primer 1FW
ccttcgcagaggaggaaact

SEQ ID NO: 2 - DNA, 20 bp, Artificial Sequence
  Chemically synthesized; primer 1RV
ctccggaaacgcattcctct

SEQ ID NO: 3 - DNA, 20 bp, Artificial Sequence
  Chemically synthesized; primer 2FW
ggtagaagttggcttagtgg

SEQ ID NO: 4 - DNA, 22 bp, Artificial Sequence
  Chemically synthesized; primer 2RV
acgcggtaaaatgatactagac

SEQ ID NO: 5 - DNA, 22 bp, Artificial Sequence
  Chemically synthesized; primer 3FW
cagttttctgtgctgagactgg

SEQ ID NO: 6 - DNA, 22 bp, Artificial Sequence
  Chemically synthesized; primer 3RV
cccacttaatggatgttcaagc